(19) United States
(12) Patent Application Publication
(10) Pub. No.: US 2026/0137950 A1
(43) Pub. Date: May 21, 2026

(54) TOOTH WHITENING SYSTEM, KIT, AND/OR METHOD

(71) Applicant: Colgate-Palmolive Company, New York, NY (US)

(72) Inventors: Leighton DAVIES-SMITH, Lebanon, NJ (US); Ruben Dario MENDEZ, Randolph, NJ (US); Brian Matthew BLOCH, Hillsborough, NJ (US)

(73) Assignee: Colgate-Palmolive Company, New York, NY (US)

(21) Appl. No.: 19/389,444

(22) Filed: Nov. 14, 2025

Related U.S. Application Data
(60) Provisional application No. 63/721,773, filed on Nov. 18, 2024.
(60) Provisional application No. 63/721,711, filed on Nov. 18, 2024.

(51) Int. Cl. A61N 5/06; A61C 19/06

(52) U.S. Cl. CPC A61N 5/0603; A61C 19/066; A61N 2005/0606; A61N 2005/0629; A61N 2005/0645; A61N 2005/0652; A61N 2005/0661; A61N 2005/0663

(57) ABSTRACT

A tooth whitening system, a tooth whitening kit, and/or a tooth whitening method. The tooth whitening system may include a whitening device and a power cord. The whitening device may include a mouthpiece, a light emitting assembly configured to emit light from the mouthpiece, and a power source that is operably coupled to the light emitting assembly. The whitening device may be activated with the power cord attached, in which case the light emitting assembly is powered by an external power supply to which the power cord is also attached. The whitening device may alternatively be powered directly by its own power source. The light emitting assembly may have a greater power output when powered by the power cord and external power supply.

### CROSS-REFERENCE TO RELATED APPLICATIONS

The present application claims the benefit of priority to United States Provisional Patent Application Serial No. 63/721,711, filed November 18, 2024, and United States Provisional Patent Application Serial No. 63/721,773, filed November 18, 2024, the entireties of which are incorporated herein by reference.

### BACKGROUND

Tooth whitening devices and systems have been in use for many years. Some such devices and systems use a combination of a whitening composition applied onto the user’s teeth followed by emitting a light onto the user’s teeth that are pre-coated with the whitening composition. Tooth whitening may be done under the supervision of a dental practitioner, or at home using a retail product. A need exists for tooth whitening systems, kits, and/or methods that provide flexibility in their use.

### BRIEF SUMMARY

The present invention is directed to a tooth whitening system, a tooth whitening kit, and/or a tooth whitening method. The tooth whitening system may include a whitening device and a power cord. The whitening device may include a mouthpiece, a light emitting assembly configured to emit light from the mouthpiece, and a power source that is operably coupled to the light emitting assembly. The whitening device may be activated with the power cord attached, in which case the light emitting assembly is powered by an external power supply to which the power cord is also attached. The whitening device may alternatively be powered directly by its own power source. The light emitting assembly may have a greater power output when powered by the power cord and external power supply.

In one aspect, the invention may be a tooth whitening system comprising: a whitening device comprising: a mouthpiece comprising a front surface; a light emitting assembly comprising one or more light emitters that are configured to generate light which is emitted from the front surface of the mouthpiece; a control circuit comprising a power source that is operably coupled to the light emitting assembly and an actuator; a power cord having a first plug configured to be operably coupled to an external power supply and a second plug configured to be operably coupled to the whitening device; wherein in a first activation state, the actuator is activated without the power cord being coupled to the whitening device and to the external power supply and the light emitting assembly is powered by the power source of the control circuit; and wherein in a second activation state, the actuator is activated with the power cord coupled to the external power supply and to the whitening device and the light emitting assembly is powered by the external power supply. In another aspect, the invention may be a tooth whitening system comprising: a whitening device comprising: a mouthpiece comprising a front surface; a light emitting assembly comprising one or more light emitters that are configured to generate light which is emitted from the front surface of the mouthpiece; a control circuit comprising a power source that is operably coupled to the light emitting assembly and an actuator; a power cord having a first plug configured to be operably coupled to an external power supply and a second plug configured to be operably coupled to the whitening device; wherein in a first activation state, the actuator is activated without the power cord being coupled to the whitening device and to the external power supply and the one or more light emitters are configured to generate the light at a first power output; wherein in a second activation state, the actuator is activated with the power cord coupled to the external power supply and to the whitening device and the one or more light emitters are configured to generate light at a second power output, the second power output being greater than the first power output.

In yet another aspect, the invention may be a tooth whitening kit comprising: a whitening device comprising: a mouthpiece comprising a front surface; a light emitting assembly comprising one or more light emitters that are configured to generate light which is emitted from the front surface of the mouthpiece; a control circuit comprising: a power source that is operably coupled to the light emitting assembly; an actuator; a first electrical contact that is operably coupled to the light emitting assembly; a second electrical contact that is operably coupled to the power source of the control circuit; and a third electrical contact that is operably coupled to ground; a power cord having a fourth electrical contact and a fifth electrical contact, the fourth and fifth electrical contacts configured to be operably coupled to the first and third electrical contacts of the control circuit, respectively, when the power cord is plugged into the whitening device; and a charging device having a sixth electrical contact and a seventh electrical contact, the sixth and seventh electrical contacts configured to be operably coupled to the second and third electrical contacts of the control circuit, respectively, when the whitening device is being charged by the charging device.

In still another aspect, the invention may be a tooth whitening kit comprising: a package containing: a tooth whitening device having a power source and one or more light emitters that are operably coupled to the power source, wherein in a first activation state the one or more light emitters are configured to be powered by the power source so that the one or more light emitters generate light; a charging device configured to be operably coupled to the tooth whitening device to charge the power source of the tooth whitening device; and a power cord configured to be operably coupled to an external power supply and to the tooth whitening device so that in a second activation state the one or more light emitters are configured to be powered by the external power supply via the power cord to generate the light while bypassing the power source.

In a further aspect, the invention may be a tooth whitening method comprising: performing a first tooth whitening session under dentist supervision, the first tooth whitening session comprising: positioning a mouthpiece of a whitening device into or adjacent to a user’s mouth; operably coupling the whitening device to an external power supply via a power cord; activating the whitening device so that light emitters of the whitening device generate light that is emitted from the mouthpiece and onto the user’s teeth, the light emitters being powered by the external power supply; and deactivating the whitening device after elapse of a first period of time; performing a second tooth whitening session without dentist supervision, the second tooth whitening session comprising: positioning the mouthpiece of the whitening device into or adjacent to the user’s mouth; activating the whitening device so that the light emitters of the whitening device generate light that is emitted from the mouthpiece and onto the user’s teeth, the light emitters being powered by a power source of the whitening device; and deactivating the whitening device after elapse of a second period of time.

In a still further aspect, the invention may be a tooth whitening system comprising: a whitening device comprising: a mouthpiece comprising a front surface and a rear surface opposite the front surface; and a handle protruding from the rear surface; and a first attachment portion; a cheek retractor assembly comprising: a cheek retractor portion that is configured to be positioned within a user’s mouth to retract the user’s cheeks; and a second attachment portion; wherein the whitening device is configured to be detachably coupled to the cheek retractor assembly via engagement between the second attachment portion of the cheek retractor assembly and the first attachment portion of the whitening device; and wherein when the whitening device is coupled to the cheek retractor assembly, relative movement between the whitening device and the cheek retractor assembly is precluded.

In yet another aspect, the invention may be a tooth whitening system comprising: a whitening device comprising: a mouthpiece comprising a front surface and a rear surface; a light emitting assembly comprising one or more light emitters that are configured to generate light which is emitted from the front surface of the mouthpiece; and a handle extending from the rear surface; a cheek retractor assembly; wherein the whitening device is configured to be detachably coupled to the cheek retractor assembly such that relative movement between the whitening device and the cheek retractor assembly is precluded.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a schematic illustration of a tooth whitening kit in accordance with an embodiment of the present invention;

FIG. 2 is a front perspective view of a tooth whitening device in accordance with an embodiment of the present invention;

FIG. 3 is a rear perspective view of the tooth whitening device of FIG. 2;

FIG. 4A is a first exploded view of the tooth whitening device of FIG. 2;

FIG. 4B is a second exploded view of the tooth whitening device of FIG. 2;

FIG. 5 is a cross-sectional view taken along line V-V of FIG. 3;

FIG. 6 is a top perspective view of a base unit of a charging device in accordance with an embodiment of the present invention;

FIG. 7 is a view illustrating the tooth whitening device being inserted into a charging receptacle of the charging device of FIG. 6;

FIG. 8 is a view illustrating the tooth whitening device located within the charging receptacle of the charging device and further illustrating a cover being attached to the base unit of the charging device;

FIG. 9 is a view illustrating the charging device with the cover attached to the base unit;

FIG. 10 is a cross-sectional view taken along line X-X of FIG. 9;

FIG. 11 is a perspective view of a power cord in accordance with an embodiment of the present invention;

FIG. 12 is a perspective view illustrating the power cord of FIG. 11 operably coupled to the tooth whitening device of FIG. 2;

FIG. 13 is a cross-sectional view taken along line XIII-XIII of FIG. 12;

FIG. 14 is a schematic electronic diagram of the tooth whitening device of FIG. 2;

FIG. 15 is a view illustrating the mouthpiece of the tooth whitening device located within the user’s mouth while being powered by a power source of the tooth whitening device;

FIG. 16 is a view illustrating a mouthpiece of the tooth whitening device located within a user’s mouth while being powered by an external power source via the power cord of FIG. 11;

FIG. 17 is a first perspective view of a cheek retractor assembly in accordance with an embodiment of the present invention;

FIG. 18 is a second perspective view of the cheek retractor assembly of FIG. 17;

FIG. 19 is an exploded view of the cheek retractor assembly illustrating a retractor component and an attachment component thereof;

FIG. 20 is another exploded view of the cheek retractor assembly illustrating the retractor component and the attachment component thereof;

FIG. 21 is a view illustrating the tooth whitening device of FIG. 2 being coupled to the cheek retractor assembly of FIG. 17;

FIG. 22 is a view illustrating the tooth whitening device of FIG. 2 coupled to the cheek retractor assembly of FIG. 17; and

FIG. 23 illustrates the cheek retractor assembly being used to retract a user’s teeth while the tooth whitening device is coupled thereto to emit light onto the user’s teeth.

### DETAILED DESCRIPTION

The following description of the preferred embodiment(s) is merely exemplary in nature and is in no way intended to limit the invention, its application, or uses.

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top” and “bottom” as well as derivatives thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments illustrating some possible non-limiting combination of features that may exist alone or in other combinations of features; the scope of the invention being defined by the claims appended hereto.

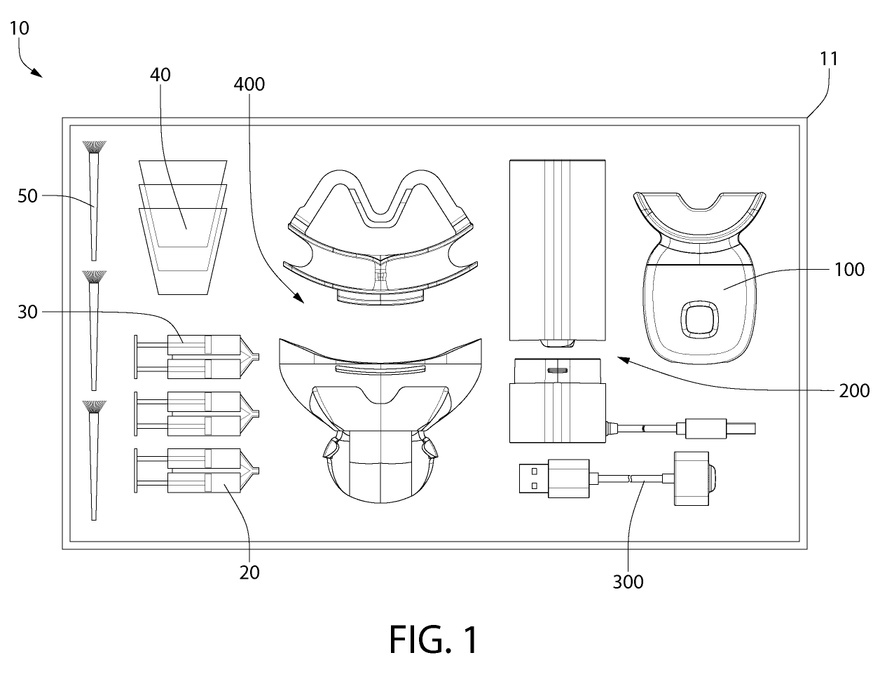

Referring to FIG. 1, a tooth whitening kit 10 is illustrated in accordance with an embodiment of the present invention. The tooth whitening kit 10 may comprise a package 11 containing various components that may be used independently or together to whiten a user’s teeth. In some embodiments, the package 11 may be a case. In other embodiments, the package 11 may be a bag, a carton, a blister, a container, or any other style of package typically used to hold multiple components together, including at a point of sale. The package 11 may also provide a way for the end user to transport all of the items of the tooth whitening kit 10 after purchase. In an embodiment, the tooth whitening kit 10 may comprise a whitening device 100, a charging device 200, a power cord 300, a cheek retractor assembly 400, one or more first dispensing units 20 that are configured to dispense a gingival barrier composition, one or more second dispensing units 30 that are configured to dispense a whitening composition, one or more containers 40, and one or more applicators 50 for applying the gingival barrier composition and/or the whitening composition onto a user’s teeth and/or soft tissue surfaces. In some embodiments, the dispensing units 20, 30, the containers, 40, and/or the applicators 50 may not be part of the tooth whitening kit 10 but may be components acquired separately from the tooth whitening kit 10 and used therewith. Variations in the specific components included in the tooth whitening kit 10 may be available and included in the scope of the invention described herein.

The one or more first dispensing units 20 may comprise a gingival barrier composition. This may be a light-cured gel that is applied by the syringe or by an applicator to the user’s gums. The gingival barrier composition may, when cured, minimize the potential for any higher level peroxide gel from contacting and irritating/burning the soft tissue. Thus, the gingival barrier composition may be used when the weight percentage of the hydrogen peroxide in the tooth whitening composition is above a certain amount, such as above 10%, or above 12%, or above 15%, or above 25%. In some embodiments, the gingival barrier composition may only be used when a tooth whitening session takes place under dental practitioner supervision. There may be any number of the first dispensing units 20 included as part of the kit 10.

The one or more second dispensing units 30 may comprise the tooth whitening composition. In an embodiment, the one or more second dispensing units 30 may be dual barrel syringes comprising two chambers. As such, the tooth whitening composition may comprise a peroxide gel in one chamber and a pH gel in the other chamber. The peroxide gel and the pH gel may be dispensed by the one or more second dispensing units 30 and then mixed together prior to application onto a user’s teeth. In some embodiments, the dispensing units 30 may comprise structures that are configured to mix the peroxide and pH gel together prior to or as they are dispensed. The tooth whitening composition may comprise any of various amounts and weight percentages of hydrogen peroxide. For example, the tooth whitening composition may comprise 6%, 10%, 15%, 20%, 25%, 30%, 40%, 50% hydrogen peroxide, or the like, in various different use cases.

The peroxide gel and the pH gel may be dispensed into one of the containers 40. As noted, the peroxide gel and the pH gel may be mixed as they are dispensed from the dispensing unit 30. In another embodiment, a user may mix the peroxide gel and the pH gel within the containers 40. Next, one of the one or more applicators 50 may be dipped into the tooth whitening composition in the container 40 and used to apply the tooth whitening composition directly onto the user’s teeth for whitening. The applicators 50 may be brush applicators, foam applicators, or any other type of applicator desired for use to apply the tooth whitening composition onto the user’s teeth.

In the exemplified embodiment, the tooth whitening composition is described as comprising a peroxide gel and a pH gel. The pH gel acts as a catalyst. In other embodiments, the pH gel may be replaced with other catalysts, including without limitation titanium dioxide, magnesium gluconate, and other heavy or semi-heavy metals.

One particular tooth whitening composition which may be used in conjunction with the whitening device 100 is described in United States Patent No. 11,040,002. However, the whitening device 100 may be used alone (i.e., without first coating the teeth with a tooth whitening composition) in some embodiments, and in other embodiments the whitening device 1000 may be used after the user coats the teeth with any tooth whitening composition, such as for example without limitation those containing hydrogen peroxide or carbamide peroxide. The tooth whitening composition which may be used in conjunction with the whitening device 100 to whiten the teeth is not limited to the specific formulation described in United States Patent No. 11,040,002 and any whitening formulation now known or later discovered which may benefit from light treatment after application to the teeth may be used. Moreover, other formulations, such as anti-sensitivity formulations, fresh breath formulations, or the like which may similarly benefit from a light treatment after application of the formulation to the teeth may be used with the whitening device 100. One additional example composition is one which includes monoperoxisulfate, which is an oxidizing agent that has a positive reaction with the light being emitted from the device. Furthermore, Vitamin C compounds acting as a reducing agent for stain removal may also be used.

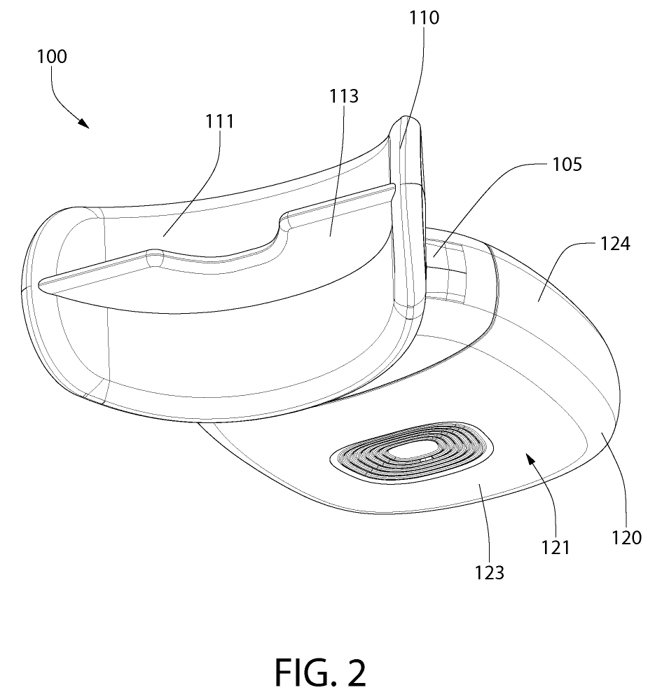

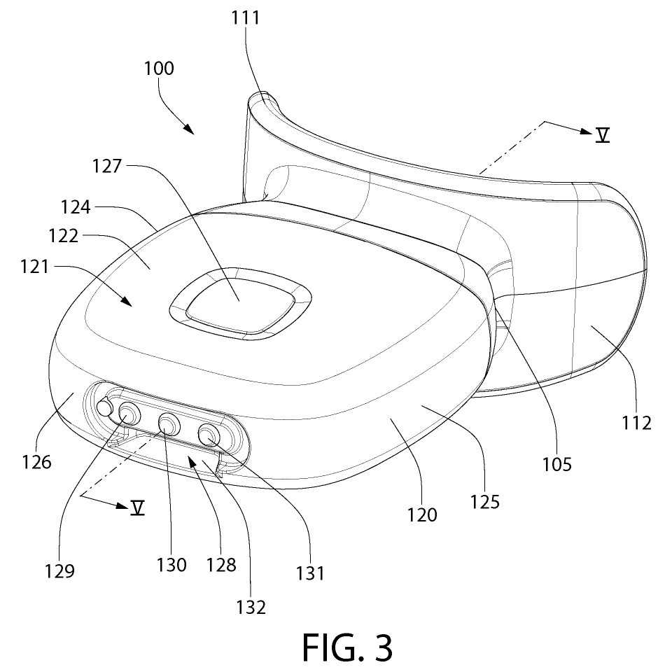

Referring now to FIGS. 2 and 3, the whitening device 100 will be described. The whitening device 100 may be the device that is configured to emit electromagnetic radiation, such as light, onto the user’s teeth. In some embodiments, the whitening device 100 may be used to emit electromagnetic radiation onto a user’s teeth in a pre-treatment step prior to applying a tooth whitening composition onto the user’s teeth, and then again after the tooth whitening composition has been applied onto the user’s teeth. In other embodiments, there may not be a pre-treatment step and the electromagnetic radiation may be applied onto the user’s teeth with the tooth whitening device 100 after the user’s teeth are coated with a tooth whitening composition.

The whitening device 100 may comprise a mouthpiece 110 and a handle 120. The mouthpiece 110 may comprise a front surface 111 through which the light is emitted and a rear surface 112 opposite the front surface 111. The front surface 111 of the mouthpiece 110 may be concave. The rear surface 112 of the mouthpiece 110 may be convex. The mouthpiece 110 may comprise a bite platform 113 protruding from the front surface 111. The bite platform 113 may be located centrally along a height of the front surface 111 of the mouthpiece 110 (measured from a bottom edge to a top edge of the front surface 111) to divide the front surface 111 into an upper portion and a lower portion that are equal in size. The upper portion of the front surface 111 may be in facing relation with the user’s upper teeth and the lower portion of the front surface 111 may be in facing relation with the user’s lower teeth during use. The bite platform 113 is depicted as a flat, plate-like structure in the exemplified embodiment. The bite platform 113 may alternatively comprise multiple, spaced apart platforms protruding from the front surface 111 of the whitening device 100. The bite platform 113 may comprise a distal wall extending from the distal end of the bite platform 113 to help maintain the mouthpiece 110 in the user’s mouth during use. Thus, variations to the structure and shape of the bite platform 113 are possible in accordance with the invention described herein. It should be noted that the term bite platform 113 does not necessarily mean that the structure is always bitten by the user during use. The bite platform 113 may be located between the user’s teeth during some use scenarios, and in other use scenarios the bite platform 113 may be located outside of the user’s mouth and not between the user’s teeth (such as when the cheek retractor assembly 400 is used, as described herein).

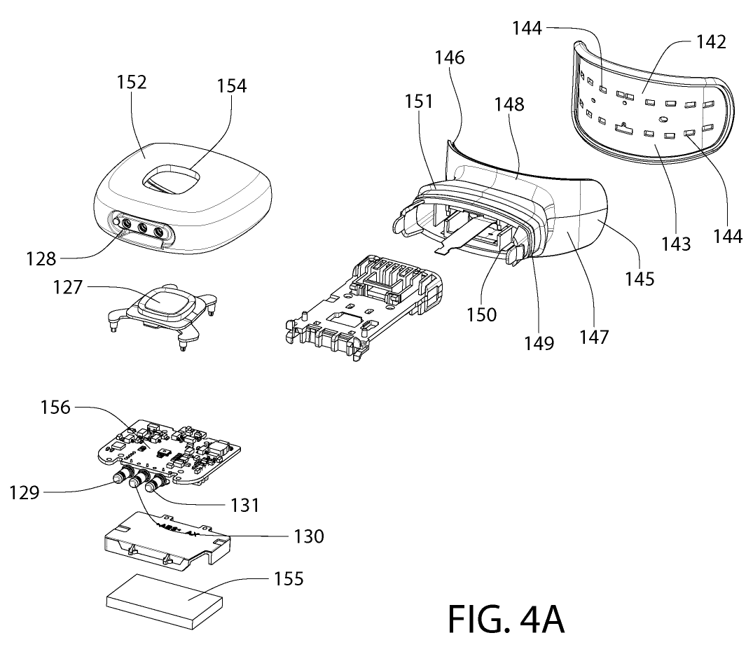

The whitening device 100 further comprises the handle 120 which protrudes or extends from the rear surface 112 of the mouthpiece 110. The handle 120 may comprise a cavity within which various electronic components of the whitening device 100 are located, as described below with reference to FIGS. 4A and 4B. The handle 120 may comprise an outer surface 121 including a top outer surface portion 122, a bottom outer surface portion 123, a first side outer surface portion 124, a second side outer surface portion 125, and a rear outer surface portion 126. The first side outer surface portion 124, the second side outer surface portion 125, and the rear outer surface portion 126 may collectively form a peripheral edge of the handle 120 and may extend between the top and bottom outer surface portions 122, 123.

The handle 120 may comprise an actuator 127. The actuator 127 may be located on or along the top outer surface portion 122 of the handle 120. Alternatively, the actuator 127 may be located along the bottom outer surface portion 123 of the handle 120. The actuator 127 may be a depressible button that, when activated, activates light emitters of the whitening device 100 to generate light and emit the light through the front surface 111 of the mouthpiece 110.

The handle 120 may further comprise a connection port 128. In the exemplified embodiment, the connection port 128 is located along the rear outer surface portion 126 of the handle 120. However, the connection port 128 may instead be located along any of the other portions of the outer surface 121 of the handle 120. The connection port 128 may be configured for coupling to the charging device 200 or the power cord 300, as described in more detail below. The connection port 128 may comprise a recess or depression in the rear outer surface portion 126 of the handle 120 (or at any other location as described above). The connection port 128 may comprise a first electrical contact 129, a second electrical contact 130, and a third electrical contact 131. The first electrical contact 129 may be operably coupled to the light emitters described below, while bypassing a power source of the whitening device 100. The second electrical contact 130 may be operably coupled to the power source of the whitening device 100. The third electrical contact 131 may be coupled to ground. This will be discussed in more detail below. The connection port 128 may further comprise an alignment notch 132. The alignment notch 132 may help to facilitate proper alignment between the whitening device 100 and the charging device 200 and/or the power cord 300 when coupled thereto.

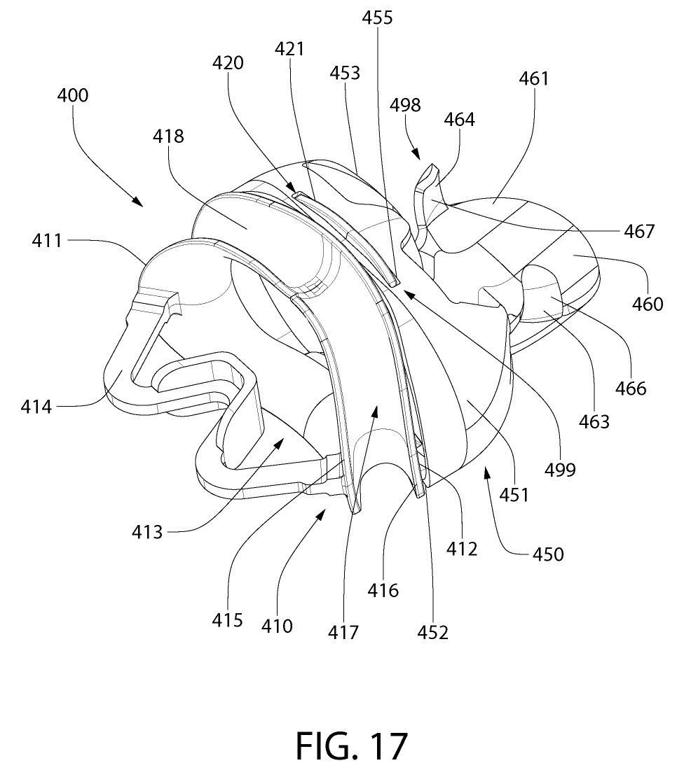

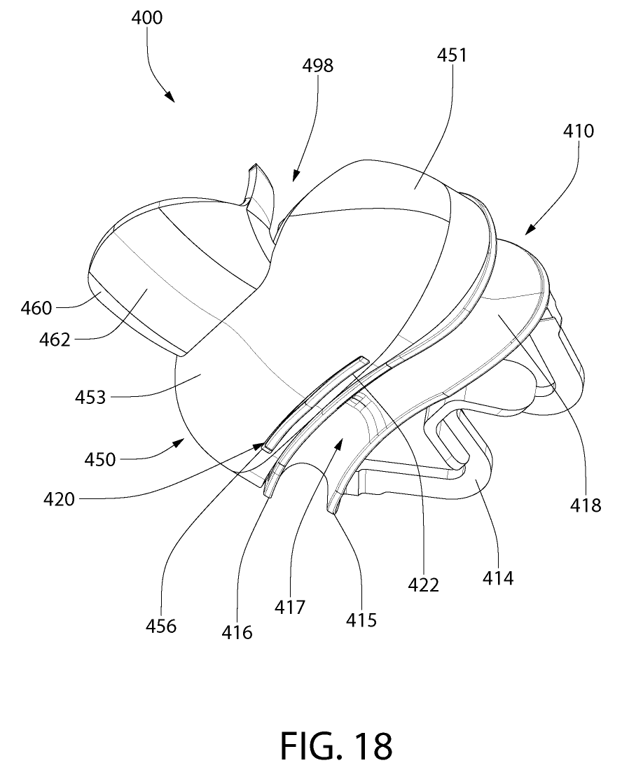

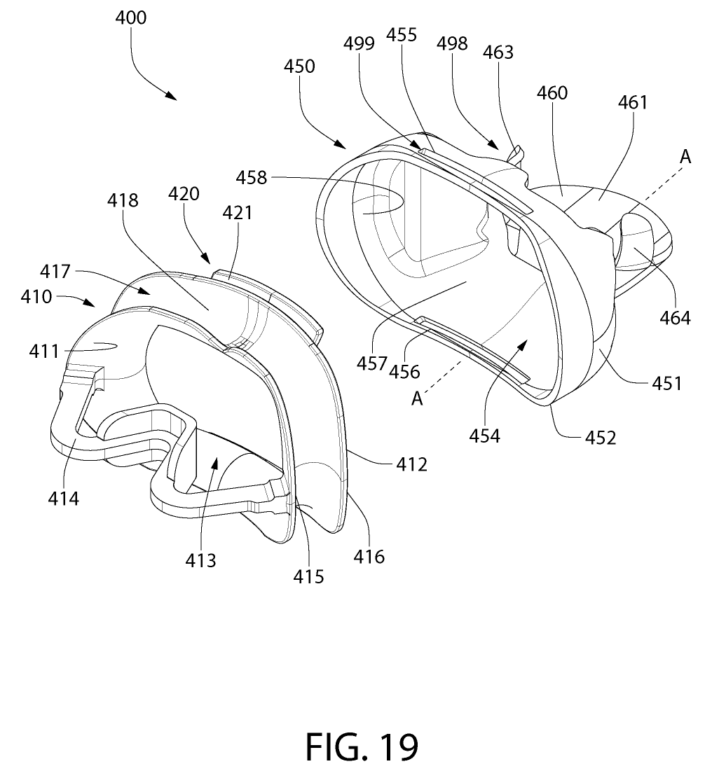

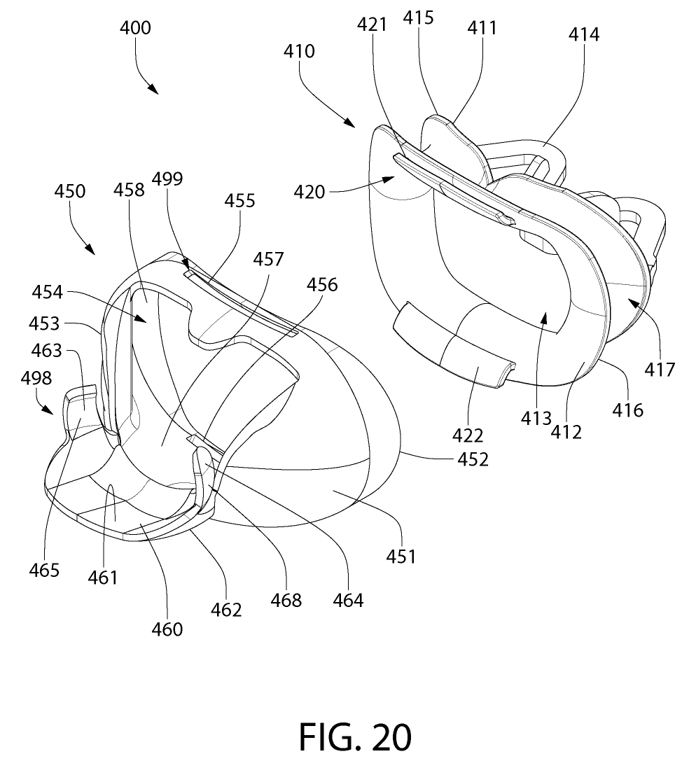

The whitening device 100 may further comprise a transition portion 105. The transition portion 105 may be formed between the handle 120 and the mouthpiece 110, or the transition portion 105 may be formed by the handle 120 and the rear surface 112 of the mouthpiece 110 at the transition between the two. The transition portion 105 may be an annular concave portion of the outer surface of the whitening device 100 which may be formed by the shapes of the handle 120 and the mouthpiece 110 at the location where the two meet. The transition portion 105 may form a first attachment portion of the whitening device 100 that facilitates a detachable coupling between the whitening device 100 and the cheek retractor assembly 400, as described below with reference to FIGS. 17-22, for example.

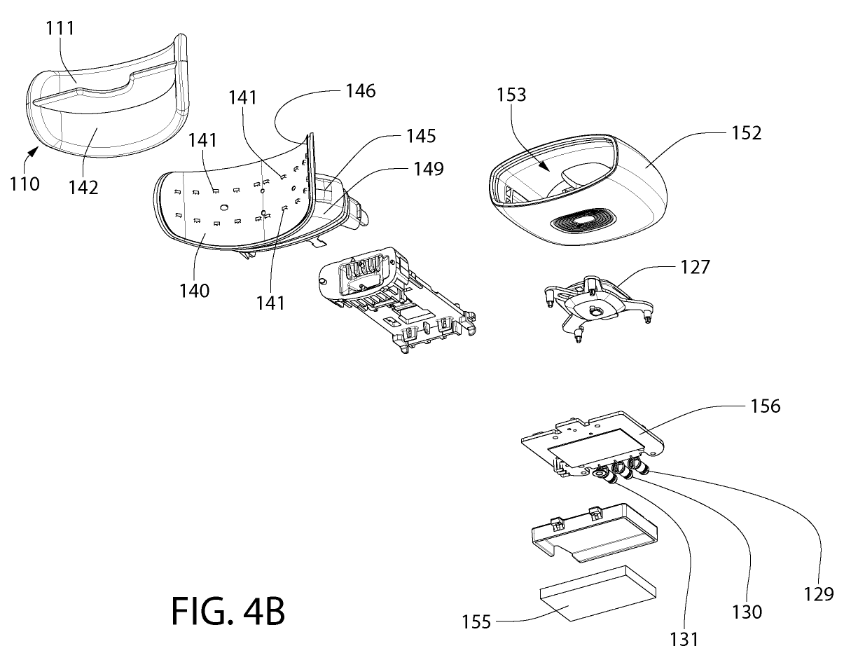

Referring to FIGS. 4A, 4B, and 5, the whitening device 100 will be further described. The whitening device 100 may comprise a light emitting assembly 140 that comprises one or more light emitters 141. In the exemplified embodiment, the one or more light emitters 141 comprises a plurality of light emitters 141. In the exemplified embodiment, the plurality of light emitters 141 are each discrete light emitting diodes or LEDs. The light emitters 141 may be arranged in rows such that an upper row of the light emitters is configured to emit light onto the user’s upper teeth while a lower row of the light emitters 141 are configured to emit light onto the user’s lower teeth. The light emitters 141 in the rows may be aligned or offset to achieve a desired effect. The light emitters 141 may be configured to generate light at a wavelength in a range between 380nm and 440nm, more specifically between 390nm and 430nm, still more specifically between 400nm and 420nm, and still more specifically between 405nm and 415nm.

The mouthpiece 110 of the whitening device 100 may further comprise a lens component (or guard component) 142 that is configured to cover the light emitting assembly 140 so that the light generated by the light emitters 141 passes through the lens component 142. The lens component 142 may comprise the front surface 111 of the mouthpiece 110. The lens component 142 may comprise a rear surface 143 with openings or recesses 144, such that each of the openings or recesses 144 is configured to receive one of the light emitters 141 therein. The lens component 142 may be formed from a transparent material so that the light generated by the light emitters 141 is configured to pass therethrough. The lens component 142 may be formed from an elastomeric material in some embodiments to enhance comfort to a user. The lens component 142 is described using the term “lens” because the light from the light emitters 141 passes therethrough. The lens component 142 does not necessarily concentrate or disperse light, although in some embodiments it may. The lens component 142 may also be referred to as a guard component because it is formed from an elastomeric material to aid in user comfort and protect the light emitters 141 located behind it.

The mouthpiece 110 may comprise a support plate 145 having an arcuate body portion 148 and a rear protrusion 149. The arcuate body portion 148 may comprise a front surface 146 and a rear surface 147, with the rear surface 147 of the arcuate body portion 148 of the support plate 145 forming the rear surface 112 of the mouthpiece 110. The light emitting assembly 140 may be positioned adjacent to the front surface 146 of the support plate 145. The light emitting assembly 140 may be sandwiched between the lens component 142 and the support plate 145 when the mouthpiece 110 is assembled.

The rear protrusion 149 may protrude from the rear surface 147 of the arcuate body portion 148 of the support plate 145. The rear protrusion 149 may define a hollow interior 150. The rear protrusion 149 may comprise one or more connection features 151. The rear protrusion 149 may comprise the transition portion 105 of the whitening device 100 described above.

The mouthpiece 110 may further comprise a housing component 152. The housing component 152 may be coupled to the rear protrusion 149 to form the handle 120. The housing component 152 may comprise one or more connection features that mate with the one or more connection features 151 of the rear protrusion 149 to facilitate the attachment. The attachment may be a snap-fit, welding, adhesion, threaded, or the like. With the housing component 152 coupled to the rear protrusion 149, the handle 120 may comprise a cavity 153. The housing component 152 may comprise a top surface that forms the top outer surface portion 122 of the handle 120. The housing component 152 may comprise an opening 154 in the top surface. The actuator 127 may be located within and/or extend through the opening 154 in the housing component 152 so that the actuator 127 is accessible to a user.

The mouthpiece 110 may further comprise a power source 155. The power source 155 may be located within the cavity 153 of the handle 120. The power source 155 may be operably coupled to the light emitting assembly 140 to power the light emitters 141 so that the light emitters 141 generate light. The power source 155 may be a battery. In some embodiments, the power source 155 may be a rechargeable battery that is rechargeable by the charging device 200 as described herein. In other embodiments, the power source 155 may be a replaceable battery that is replaceable by an end user. The mouthpiece 110 may further comprise a circuit board 156 with additional electronic components that facilitate the functionality and operation of the mouthpiece 110. The circuit board 156 may comprise any of one or more of a processor, a memory, a timer, a charging circuit, a first current drive circuit, and a second current drive circuit, some of which will be discussed in additional detail below. The circuit board 156 may be located within the cavity 153 of the handle 120. Thus, the mouthpiece 110 may comprise a control circuit that comprises the power source 155, the actuator 127, and any of the additional electronic components located on the circuit board 156. In an embodiment, the light emitting assembly 140 may also form part of the control circuit.

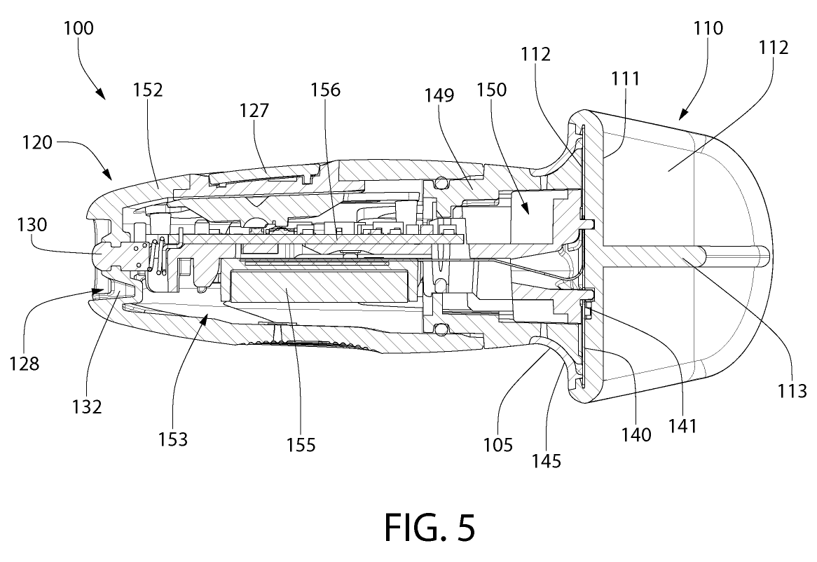

Referring to FIG. 6, a base unit 210 of the charging device 200 will be described. The base unit 210 comprises a housing 211 having a bottom end 211 and a top end 212. The base unit 210 further comprises a charger cord 213 operably coupled thereto, with the charger cord 213 extending to a distal end comprising a charger plug 214. The charger plug 214 is illustrated as a USB port in the exemplified embodiment, but may take other forms including being a standard/conventional electric plug intended to plug into a mains power supply. The base unit 210 comprises a receptacle (i.e., charging receptacle) 215 that extends from a floor 216 to an opening 217 that is located in the top end 212 of the housing 211. The receptacle 215 is configured to receive at least a portion of the whitening device 100 to charge the power source 155 of the whitening device 100, as described in more detail below.

The base unit 210 of the charging device 200 may comprise electrical contacts that are configured to become operably coupled to the second and third electrical contacts 130, 131 of the whitening device 100 when the whitening device 100 is properly positioned within the receptacle 215. Specifically, the base unit 210 of the charging device 200 may comprise a sixth electrical contact 218 and a seventh electrical contact 219. The sixth and seventh electrical contacts 218, 219 may be located along the floor 216 of the receptacle 215. All of the electrical contacts described herein may be formed from metal or other electrically conductive material to permit electrical connectivity between various components when coupled together.

The base unit 210 of the charging device 200 may further comprise an alignment tab 220 protruding from the floor 216 of the receptacle 215. The alignment tab 220 is positioned adjacent to the sixth electrical contact 218 in the exemplified embodiment, but may be located elsewhere in other embodiments. The alignment tab 220 may be configured to mate with the alignment notch 132 of the whitening device 100 as mentioned above to ensure that the whitening device 100 is properly positioned within he receptacle 215 for charging. For example, the alignment tab 220 will prevent the whitening device 100 from being positioned in the receptacle 215 in any orientation other than the one where the alignment tab 220 nests within the alignment notch 132. This ensures that the sixth and seventh electrical contacts 218, 219 of the charging unit 200 mate with the second and third electrical contacts 130, 131 of the whitening device 100 when the whitening device 100 is located in the receptacle 215 to facilitate charging of the power source 155 of the whitening device 100.

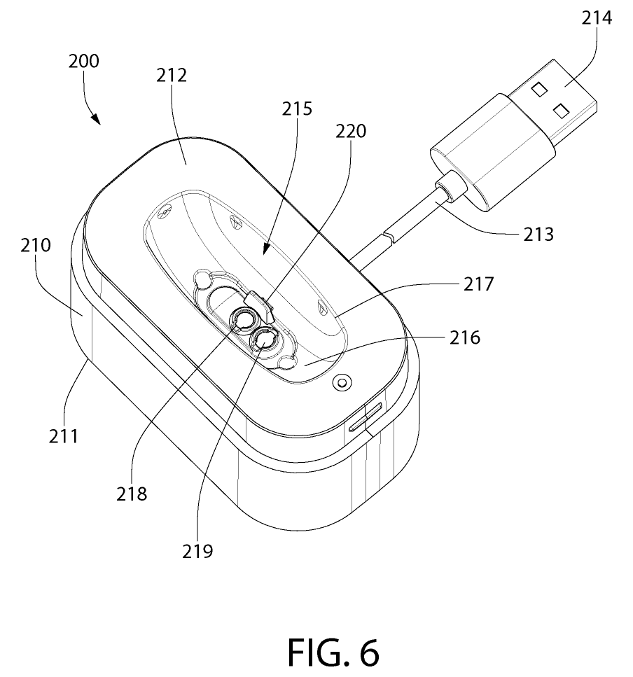

Referring to FIG. 7, the whitening device 100 is illustrated being inserted into the receptacle 215 of the base unit 210 of the charging device 200. Specifically, the whitening device 100 is configured to be inserted into the receptacle 215 of the base unit 210 with the rear outer surface portion 126 of the handle 120 entering the receptacle 215 first. This ensures that the second and third electrical contacts 130, 131 of the whitening device 100 are able to come into electrical contact with the sixth and seventh electrical contacts 218, 219 of the charging device 200.

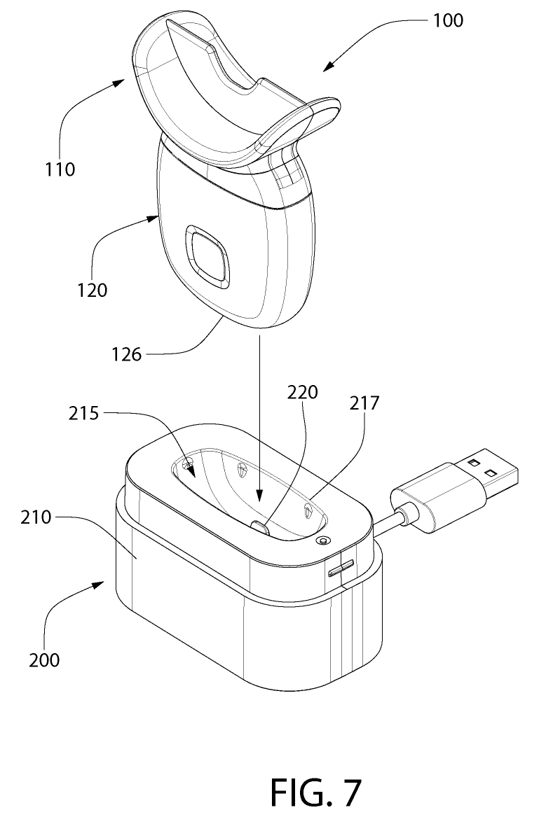

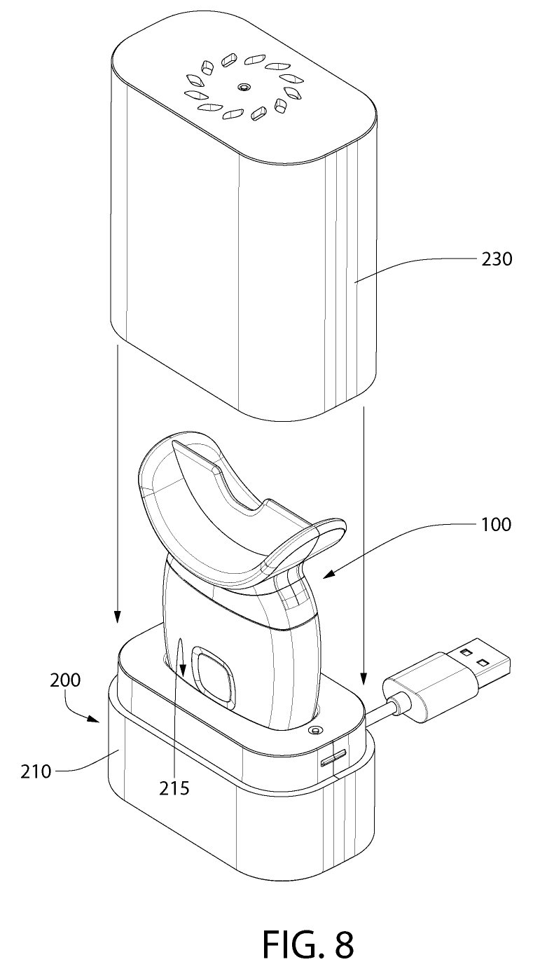

FIG. 8 illustrates the whitening device 100 fully positioned within the receptacle 215 of the base unit 210 of the charging device 200. FIG. 8 further illustrates a cover 230 of the charging device 200 being positioned for attachment to the base unit 210 of the charging device 200. The cover 230 may define an interior cavity so that while a portion of the whitening device 100 is located within he receptacle 215, a remainder of the whitening device 100 is located in the interior cavity of the cover 230. FIG. 9 illustrates the charging device 200 with the cover 230 coupled to the base unit 210 to fully enclose the whitening device 100 therein. The cover 230 may have a top surface 231 with apertures 232 formed therein to allow air flow into and out of the interior of the charging device 200.

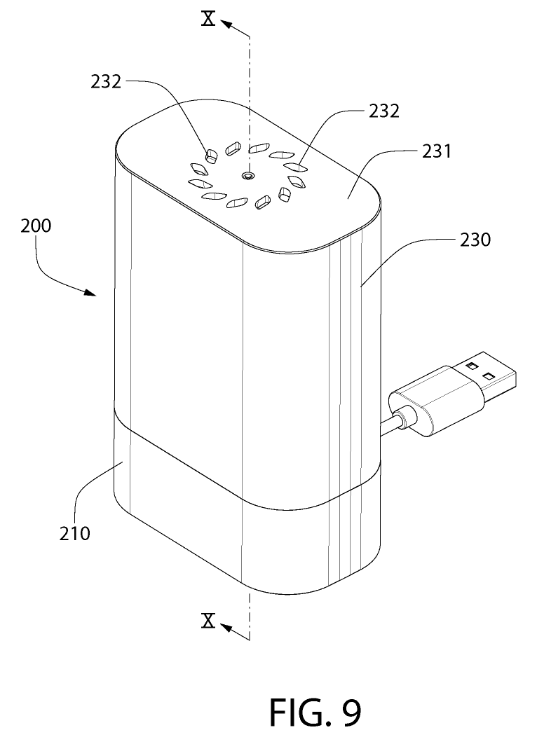

Referring to FIG. 10, a cross-sectional view is provided showing the whitening device 100 properly seated within the receptacle 215 of the base unit 210 of the charging device 200 with the cover 230 attached to the base unit 210 to define an enclosed interior. As depicted, the sixth electrical contact 218 of the charging device 200 is electrically coupled to the second electrical contact 130 of the whitening device 100 and the seventh electrical contact 219 of the charging device 200 is electrically coupled to the third electrical contact 131 of the whitening device 100. The electrical coupling may be achieved by a direct physical contact between the different electrical contacts 218/130, 219/131. Alternatively, electrical coupling may be achieved in other ways, including inductively or the like.

As noted above, the second electrical contact 130 of the whitening device 100 is operably coupled to the power source 155 and the third electrical contact 131 of the whitening device 100 is operably coupled to ground. Thus, the connection of the sixth and seventh electrical contacts 218, 219 of the charging unit 200 to the second and third electrical contacts 130, 131 of the whitening device 100 allows the charging unit 200 to charge the power source 155 of the whitening device 100. Specifically, the charger plug 214 of the charger cord 213 may be plugged into a power source, which may be a mains power supply such as a wall outlet, or may be an electronic device such as a smart phone or a computer. The power from the mains power supply or electronic device may then be transmitted to the power source 155 of the whitening device 100 to charge the power source 155 to allow the power source 155 to have sufficient power to power the light emitters to allow for plug-free operation.

In the exemplified embodiment, the cover 230 comprises a pair of retention protrusions 241 extending downwardly from an interior of a top part of the cover 230 and/or from a top portion of a sidewall of the cover 230. The retention protrusions 241 may contact the whitening device 100 when the cover 230 is secured in place on the base unit 210, thereby forcing the whitening device 100 downwardly within the receptacle 215. The retention protrusions 241 may therefore ensure that the whitening device 100 is properly seated within the receptacle 215 so that the sixth electrical contact 218 of the charging device 200 contacts the second electrical contact 130 of the whitening device 100 and the seventh electrical contact 219 of the charging device 200 contacts the third electrical contact 131 of the whitening device 100.

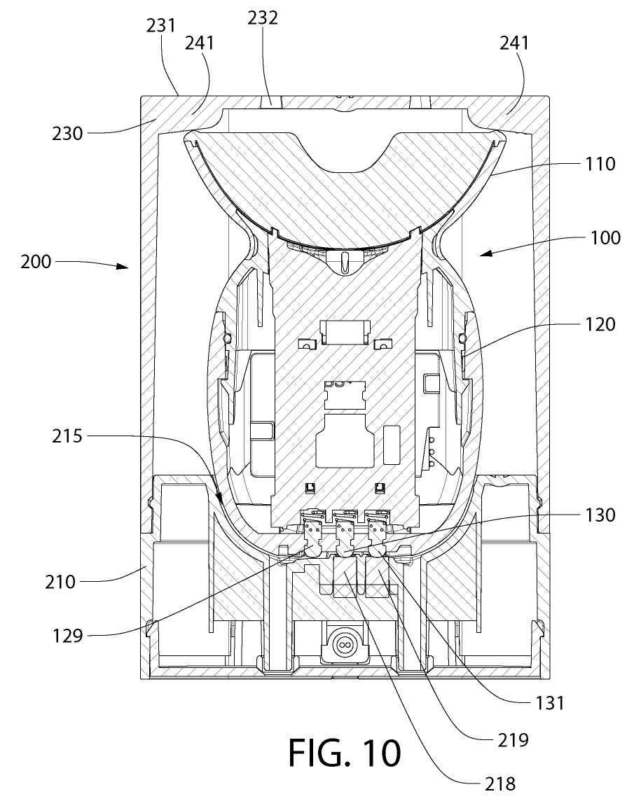

Referring to FIG. 11, the power cord 300 is illustrated. The power cord 300 comprises a first plug 310, a second plug 320, and a cord portion 330 extending between the first and second plugs 310, 320. The cord portion 330 may comprise a sheath and wires that are enclosed within the sheath. The first plug 310 may be configured for operable coupling to an external power supply, such as a mains power supply or an external electronic device such as a smart phone, a computer, a laptop, a portable power station, or the like. In the exemplified embodiment, the first plug 310 is a USB connector type. However, the invention is not to be so limited and the first plug 310 may alternatively comprise an electrical connector type, such as one configured for direct attachment to a wall outlet and a mains power supply. The second plug 320 may be configured for operable coupling to the whitening device 100. The second plug 320 may comprise an alignment tab 321 that is configured to mate with the alignment notch 132 of the whitening device 100 described above to ensure proper alignment of the second plug 320 with the connection port 128 of the whitening device 100. The second plug 320 may further comprise a fourth electrical contact 322 and a fifth electrical contact 323. The fourth and fifth electrical contacts 322, 323 may be configured to mate with or be in contact with the first and third electrical contacts 129, 131 of the whitening device 100 when the second plug 320 is plugged into the connection port 128 of the whitening device 100.

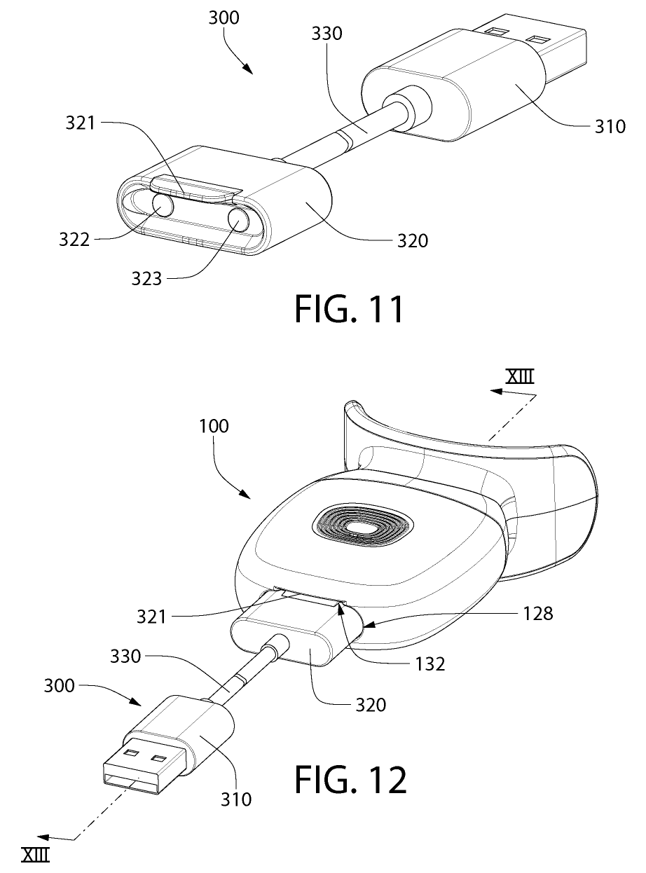

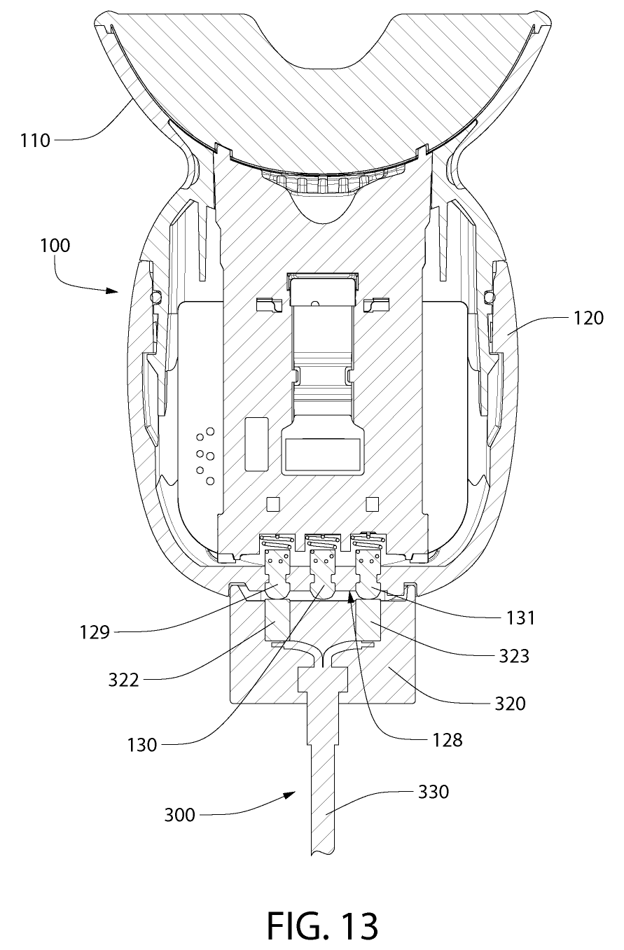

Referring to FIGS. 12 and 13, the power cord 300 is illustrated with the second plug 320 plugged into the connection port 128 of the whitening device 100. While not shown in this view, the first plug 310 of the power cord 300 may be plugged into an external power supply (i.e., mains power supply, external electronic device, or the like) so that power from the external power supply may be transmitted to the whitening device 100. When the second plug 320 is plugged into the connection port 128 of the whitening device 100, the fourth electrical contact 322 of the power cord 300 is electrically coupled to the first electrical contact 129 of the whitening device 100 and the fifth electrical contact 322 of the power cord 300 is electrically coupled to the third electrical contact 131 of the whitening device 100. As shown, the electrical coupling may be achieved by a direct physical contact between the first and fourth electrical contacts 129, 322 and between the third and fifth electrical contacts 131, 323. Alternatively, the electrical coupling may be an inductive coupling or some other wireless or non-contact type coupling to enable power transmission as described herein.

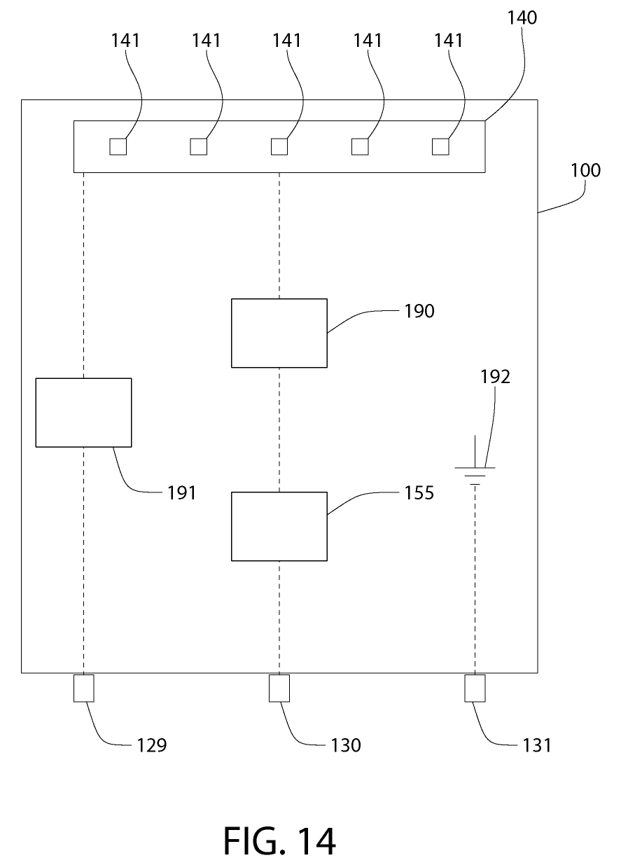

Referring briefly to FIG. 14, a schematic electric diagram of the whitening device 100 is provided. The first electrical contact 129 is operably coupled to the light emitting assembly 140 via a second current drive circuit 191. The second electrical contact 130 is operably coupled to the light emitting assembly 140 via the power source 155 and a first current drive circuit 190. The third electrical contact 131 is operably coupled to ground 192.

If the whitening device 100 is activated (such as by activating the actuator 127) while the power cord 300 is not coupled to the whitening device 100 and/or to the external electronic device, the power is transmitted form the power source 155 to the light emitting assembly 140 to power the light emitters 141. Specifically, the power is transmitted from the power source 155, through the first current drive circuit 190, and to the light emitting assembly 140. When the power is transmitted from the first current drive circuit 190, a first current is provided /transmitted to the one or more light emitters. Furthermore, when the power is transmitted from the power source 155 to the light emitters 141 via the first current drive circuit 190, the light emitters generate the light at a first power output.

When the power cord 300 is coupled to the whitening device 100, the fourth electrical contact 322 of the power cord 300 is electrically coupled to the first electrical contact 129 of the whitening device 100 and the fifth electrical contact 323 of the power cord 300 is electrically coupled to the second electrical contact 131 of the whitening device 100. As such, if the whitening device 100 is activated (such as by activating the actuator 127) while the power cord 300 is coupled to the whitening device 100 and to an external electronic device, the power is transmitted from the external electronic device to the light assembly 140 while bypassing the power source 155. Thus, when the power cord 300 is coupled to the whitening device 100 and to the external electronic device, all power transmitted to the light assembly 140 is transmitted directly from the external electronic device with no power being transmitted from the power source 155. Furthermore, the power cord 300 does not charge or otherwise supply power to the power source 155 since the power cord 300 does not have an electrical contact that is electrically coupled to the second electrical contact 130 of the whitening device 100.

When the power cord 300 is coupled to the whitening device 100 and to an external electronic device, the power is transmitted through the second current drive circuit 191. The second current drive circuit 191 may be configured to provide a second current to the one or more light emitters 141 of the light assembly 140. When the light emitters 141 are driven by the second current drive circuit 191, the light emitters 141 may be configured to generate light at a second power output.

In the exemplified embodiment, the second current may be greater than the first current and the second power output may be greater than the first power output. Thus, in an embodiment, when the power cord 300 is electrically coupled to the whitening device and power is transmitted from an external power supply to the light emitters 141 via the second current drive circuit, the light emitters 141 are driven with a higher current and have a higher power output than when the power cord 300 is not used. In one embodiment, the first current provided to the light emitters 141 when the power cord 300 is not used may be 6mA, or 10mA, or 12mA. In one embodiment, the second current provided to the light emitters 141 when the power cord 300 is used may be 14mA, 16mA, 18mA, 20mA, or more than 20mA. In an embodiment, the lower first current may be less than 14mA, more specifically approximately 6-12mA, and the higher second current may be greater than or equal to 14mA, or more specifically between approximately 14mA and approximately 20mA, or greater than 20mA (the term approximately as used here allowing for a tolerance of plus or minus 5%). In some embodiments, the second current provided to the light emitters 141 when the power cord 300 is used may be up to 25mA, or 30mA, or 35mA, or 40mA, or 45mA, or 50mA, or more than 50mA. In some embodiments, higher current may result in faster tooth whitening, although this must be balanced against other factors such as sensitivity, irritation, and pain for the user.

In an embodiment, the second power output may comprise a radiant flux of approximately 0.2 Watts and the first power output may comprise a radiant flux of approximately 0.12 Watts. In an embodiment, the light emitters 141 may have a luminous flux of approximately 4.2 Lumens when the second current is provided and a luminous flux of approximately 2.3 when the first current is provided. Thus, when the power cord 300 is used during a treatment with the whitening device 100, the light emitters 141 have a higher radiant flux, a higher power output, and a higher luminous flux as compared to the use of the whitening device 100 without the power cord 300. The term “approximately” as used in this paragraph may include a tolerance of plus/minus 10%).

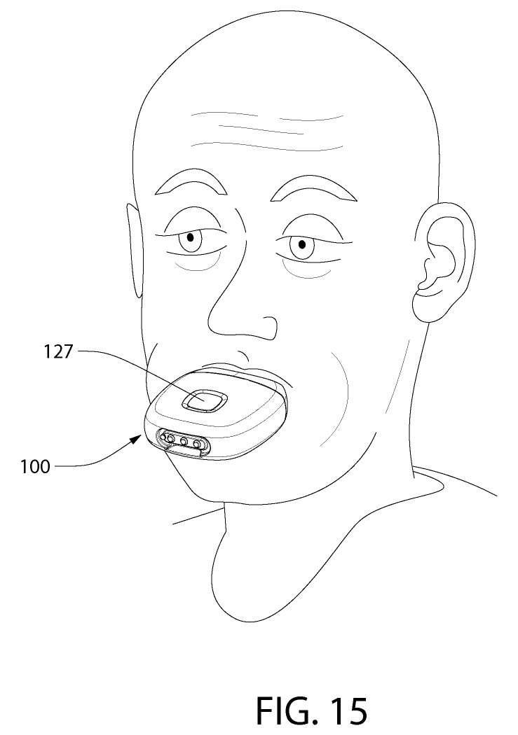

Referring to FIG. 15, the whitening device 100 is illustrated in use to whiten a user’s teeth. The mouthpiece 110 of the whitening device 100 may be positioned in the user’s mouth so that the front surface 111 of the mouthpiece 110 faces the front surfaces of the user’s teeth. The handle 120 of the whitening device 100 is located external to the user’s mouth. In FIG. 15, the power cord 300 is not coupled to the whitening device 100. Thus, the whitening device 100 is powering the light emitters 141 from the power source 155 that forms part of the whitening device 100 (i.e., the onboard power source or batteries 155). The power source 155 may need to be charged before use by the charging unit 200 as described herein. When fully charged, the power source 155 may have sufficient charge for use during three or four or five or six or the like ten minute whitening sessions. In some embodiments, prior to inserting the mouthpiece 110 of the whitening device 100 into the user’s mouth, the user may apply a whitening composition, such as a hydrogen-peroxide containing whitening composition, onto the front surfaces of the teeth. In other embodiments, the user may perform a pre-treatment step by inserting the mouthpiece 110 of the whitening device 100 into the user’s mouth and activating the whitening device 100 to emit the light onto the user’s teeth for a time period prior to applying the whitening composition onto the user’s teeth. In such embodiment, the mouthpiece 110 of the whitening device 100 will be removed from the user’s mouth after the pre-treatment step, then the whitening composition will be applied onto the user’s teeth, and then the mouthpiece 110 will be reinserted into the user’s mouth for another light treatment on the teeth coated with the whitening composition. The whitening device 100 may then be used for a desired period of time to emit light at a wavelength in the range described above onto the teeth that are pre-coated with the whitening composition.

FIG. 15 illustrates the whitening device 100 being used in a first activation state. Specifically, when the actuator 127 is activated without the power cord 300 being coupled to the whitening device 100 and/or to the external power supply, the light emitting assembly 140 is powered by the power source 155 of the whitening device 100. This results in the light emitters 141 being powered at the first current level and emitting light at the first power output level. The whitening device 100 may be operated in the first activation state so long as the power source 155 has sufficient charge to power the light emitters 141 at the desired current.

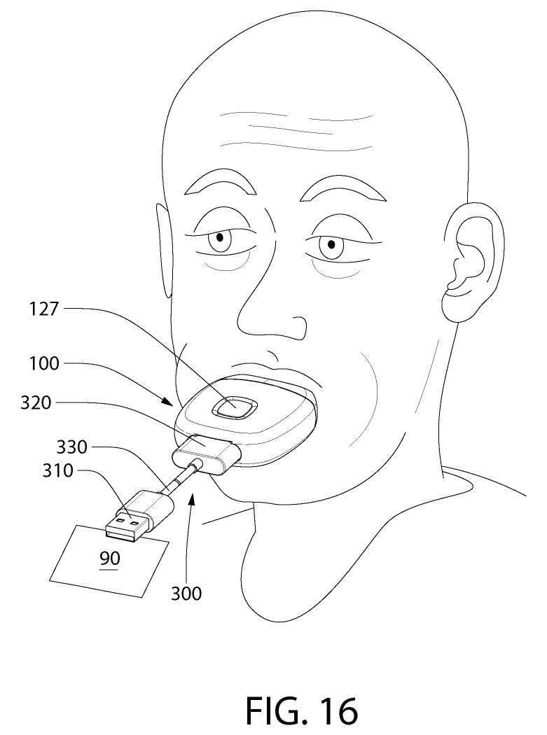

FIG. 16 illustrates the whitening device 100 being used in a second activation state, whereby the power cord 300 is operably coupled to the whitening device 100 and to an external power supply 90. When the actuator 127 is activated while the power cord 300 is coupled to the whitening device 100 and to the external electronic device 90, the light emitting assembly 140 is powered by the external power supply 90. This results in the light emitters 141 being powered at the second current level (which is greater than the first current level) and emitting light at the second power output level (which is greater than the first power output level).

In some embodiments, the whitening device 100 may only be able to be activated in the second activation state when under the supervision of a dental practitioner. Specifically, the power cord 300 may be accessible only to the dental practitioner, due to the whitening device 100 operating at a higher current and power output when the power cord 300 is used. The higher current and power output levels possible when the power cord 300 is used could subject the user to greater risk of damage to the soft tissue/gums/teeth owing to those greater power outputs. Thus, in some embodiments the power cord 300 may only be accessible to the dental practitioner so that the whitening device 100 is only capable of reaching those higher current and power output levels when the patient is under the care of a dental practitioner. In such embodiments, the whitening device 100 may be used with the power cord 300 under the care of a dental practitioner, and then the whitening device 100 may be taken home and used at home by the user without the power cord 300, with the onboard power source 155 of the whitening device 100 powering the light emitters 141 as described herein.

As discussed above, when the power cord 300 is used to power the light emitters 141 with an external power supply (such as a mains power supply), the onboard power source 155 of the whitening device 100 is bypassed such that it is neither charged by the external power supply nor used to supply power to the light emitters 141. When the power cord 300 is not used to power the light emitters 141, the light emitters 141 are powered by the onboard power source 155 so long as the power source 155 has sufficient charge to power the light emitters 141. The power source 155 may be charged by the charging device 200 as described above. In some embodiments, only one of the power cord 300 and the charging device 200 may be operably coupled to the whitening device 100 at any given time since both are plugged into the same connection port 128 of the whitening device 100. In an alternative embodiment, the whitening device 100 may comprise two separate connection ports so that the power cord 300 may be plugged into one of the connection ports and the charging device 200 may be plugged into the other one of the connection ports.

The availability of the power cord 300 to the dental practitioner may allow a dental practitioner to avoid having to remember to charge the whitening device 100 prior to first use and use each day. The availability of the power cord 300 to the dental practitioner may also allow the dental practitioner to use the whitening device 100 for longer whitening sessions that may be possible when the whitening device 100 is powered only by the onboard power source 155. Furthermore, the availability of the power cord 300 to the dental practitioner may allow the dental practitioner to offer a higher benefit treatment due to the whitening device 100 providing a higher current to the light emitters 141 and the light emitters 141 generating light at a higher power output when the whitening device 100 is used with the power cord 300 compared to being used without the power cord 300.

In some embodiments, the whitening device 100 may have a timer. In some embodiments, the timer may be set to power off the whitening device 100 after ten minutes of operation so that the whitening device 100 is programmed to operate in ten minute sessions. Some patients may be able to tolerate longer sessions and may benefit from them. Thus, the timer may be user configurable so that the user can decide how long the whitening session should be. In some embodiments, the length of the whitening session may be dependent on the whitening composition being used. Thus, optimized configurations may be used by modifying the power output levels (by activating with or without the power cord 300), light-on time for each session, and/or hydrogen peroxide weight percentages in the whitening composition. In some embodiments, when the power cord 300 is coupled to the whitening device 100 during a treatment session, the timer may not be operational. Since the power cord 300 may only be used by a dental practitioner in some embodiments, the dental practitioner will be able to determine the appropriate treatment session time such that the timer may not be required. The timer may be operational when the whitening device 100 is used by the user without dental practitioner oversight and without the power cord 300 attached.

In some embodiments, the invention may be directed to a tooth whitening method. The tooth whitening method may comprise performing a first tooth whitening session under dentist supervision followed by later performing a second tooth whitening session without dental supervision (such as at home). The first tooth whitening session may comprise positioning the mouthpiece 110 of the whitening device 100 into or adjacent to a user’s mouth while the whitening device 100 is operably coupled to the external power supply 90 via the power cord 300. The mouthpiece 110 may be located directly inside of the user’s mouth, or may be coupled to a cheek retractor as discussed below with reference to FIGS. 17-25 such that the mouthpiece 110 is adjacent to the user’s mouth but not positioned inside the user’s mouth. Next, the whitening device 100 is activated so that the light emitters 140 of the whitening device 100 generate light that is emitted from the mouthpiece 100 and onto the user’s teeth. The light emitters in this first tooth whitening session are powered by the external power supply 90. After a sufficient amount of time has passed, the whitening device 100 may be deactivated. The deactivation may occur automatically or may be user-initiated.

The first tooth whitening session may take place at a dentist’s office. The user may take the whitening device 100 home to perform second and subsequent tooth whitening sessions. For example, in a second tooth whitening session at home and not under dentist supervision, the mouthpiece 100 of the whitening device 100 may be positioned into or adjacent to the user’s mouth. Next, the whitening device 100 may be activated so that the light emitters 141 of the whitening device 100 generate light that is emitted from the mouthpiece 110 and onto the user’s teeth. During this sections tooth whitening session, the light emitters 141 may be powered by the power source 155 of the whitening device 100, which may be an onboard power source. Finally, the whitening device 100 may be deactivated (automatically or by user initiation) after elapse of a desired amount of time.

As discussed herein, since the power cord 300 is used during the first tooth whitening session, the light emitters 141 may be driven at a higher current and with a higher power output as compared to the second tooth whitening session when the power cord 300 is not used. In each of the first and second tooth whitening sessions, a whitening composition, such as a hydrogen- peroxide containing composition, is preferably applied to the user’s teeth prior to emitting the light onto the user’s teeth.

Referring to FIGS. 17-20, the cheek retractor assembly 400 will be further described. In some embodiments, the cheek retractor assembly 400 may be used with the whitening device 100 in the second activation state wherein the power cord 300 is operably coupled to the whitening device 100 and the light emitters 141 have the relatively higher power output (as compared to operation of the light emitters 141 when the power cord 300 is not operably coupled to the whitening device 100 as described above). For example, in some embodiments when the whitening device 16 is used as shown in FIG. 16, the whitening device 100 may be coupled to the cheek retractor assembly 400 which is at least partially positioned in the user’s mouth. In other embodiments, the cheek retractor assembly 400 may be used under either of the first and second activation states, under dental supervision or not.

In some use scenarios, higher levels of hydrogen peroxide are used in the whitening process when done under the supervision of a dental practitioner than when done at home without the supervision of a dental practitioner. This may be due to local laws or for other reasons. In some embodiments, dental professionals may use higher levels of hydrogen peroxide in the whitening composition in order to quickly deliver the best whitening outcome, while providing necessary safety precautions and procedures to eliminate or reduce risks to the patient in terms of discomfort or pain and to protect the user’s other oral surfaces like the gums, soft tissues, and the like. In some embodiments, when tooth whitening is done under dental supervision, part of the whitening treatment process may involve the use of the cheek retractor assembly 400 (with or without lip protection) in order to provide the dental practitioner with full visibility and access to the ‘smile’ teeth. To protect the soft tissue (e.g., gums) from exposure to the relatively higher concentration bleaching agent (e.g., hydrogen peroxide), dental practitioners may use a gingival barrier or dam - typically a light-cured gel applied by syringe to the gums which, when cured, minimizes the potential for the higher level peroxide gel from contacting and irritating/burning the soft tissue.

In the exemplified embodiment, the cheek retractor assembly 400 comprises a retractor component 410 and an attachment component 450 that are detachably coupled together. In other embodiments, the retractor component 410 and the attachment component 450 may be part of an integral, monolithic structure. That is, in an alternative embodiment the cheek retractor assembly 400 may be a unitary structure comprising all features of the retractor component 410 and the attachment component 450 as described herein with the exception of features permitting the detachable coupling of those components to one another. Further modifications to the cheek retractor assembly 400 may be included within the scope of the invention described herein, some of which is discussed below. The details of the cheek retractor assembly 400 will be discussed below with reference to the retractor component 410 and the attachment component 450, but it should be appreciated that the description would be applicable to the cheek retractor assembly 400 even if formed as a monolithic part.

The retractor component 410 comprises a first end 411, a second end 412 opposite the first end 411, and a through-hole 413 that extends from the first end 411 to the second end 412. The through-hole 413 provides visual access to the user’s smile teeth (the teeth of the user that are visible when smiling) even when the retractor component 410 is located within the user’s mouth as described herein. In the exemplified embodiment, the retractor component 410 comprises a tongue guard 414 extending from the first end 411. The tongue guard 414 may be used to keep the user’s tongue stationary within the user’s mouth during treatment. The tongue guard 414 is illustrated with a “W” shaped structure, although the invention is not to be so limited and the tongue guard 414 may take on other shapes as desired. In some embodiments, the tongue guard 414 may be altogether omitted.

The retractor component 410 may comprise a first annular flange portion 415 that comprises the first end 411, a second annular flange portion 416 that comprises the second end, and a channel 417 having a floor 418 that extends between the first and second annular flange portions 415, 416. When in use, the user’s cheeks are positioned within the channel 417 with the first annular flange portion 415 located internally in the user’s mouth and abutting against the inside surfaces of the user’s cheeks and the second annular flange portion 416 abutting against the outside surfaces of the user’s cheeks. The retractor component 410 thereby retains the user’s cheeks in a retracted state whereby the user’s cheeks are pulled away from the user’s teeth so that the teeth are visible and accessible for treatment. The tongue guard 414 may be positioned adjacent to a top surface of the user’s tongue to restrain the tongue within the lower part of the user’s mouth. The first and second annular flange portions 415, 416, the channel 417, and the tongue guard 414 may collectively form a cheek retractor portion of the retractor component 410 of the cheek retractor assembly 400.

The retractor component 410 comprises at least one coupling element 420 that is configured to facilitate the detachable coupling of the retractor component 410 to the attachment component 450. In the exemplified embodiment, the at least one coupling element comprises a first tab 421 protruding from the second end 412 of the retractor component 410 and a second tab 422 protruding from the second end 412 of the retractor component 410. In the exemplified embodiment, the first tab 421 is located along a top portion of the second end 412 of the retractor component 410 and the second tab 422 is located along a bottom portion of the second end 412 of the retractor component 410. However, the invention is not to be so limited and, in another embodiment, the first tab 421 may be located along a first side portion of the second end 412 of the retractor component 410 and the second tab 422 may be located along a second side portion of the second end 412 of the retractor component 410. In yet another embodiment, there may be four of the tabs, each protruding from one of the top, bottom, first side, and second side portions of the second end 412 of the retractor component 410. The first and second tabs 421, 422 may be referred to herein as a pair of tabs. While the first and second tabs 421, 422 are illustrated as forming the at least one coupling element 420 of the retractor component 410 in the exemplified embodiment, in an alternative embodiment the at least one coupling element 420 may comprise a pair of slots instead of tabs. Specifically, either the retractor component 410 comprises a pair of tabs and the attachment component 450 comprises a pair of slots as shown, or the retractor component 410 comprises a pair of slots and the attachment component 450 comprises a pair of tabs. Other structures may be used to facilitate the attachment of the retractor component 410 and the attachment component 450 (aside from tabs/slots) in other embodiments.

In the exemplified embodiment, the first and second tabs 421, 422 are elongated in a direction perpendicular to the through-hole 413 (specifically, to an axis of the through-hole 413). Furthermore, the first and second tabs 421, 422 may have convex inner surfaces and concave outer surfaces. The convex inner surfaces of the first and second tabs 421, 422 may generally face one another. The specific structure, shape, number, and positioning of the tabs 421, 422 is not to be limiting of the invention in all embodiments. For example, the tabs 421, 422 could be L-shaped tabs rather than arcuate shaped tabs while still achieving the attachment/detachment functionality as described herein.

The attachment component 450 of the cheek retractor assembly 400 is configured to be detachably coupled to the retractor component 410 and also to the whitening device 100, as described herein. Thus, the attachment component 450 comprises an attachment portion 498 that is configured to facilitate an attachment of the attachment component 450 to the whitening device 100 and at least one second coupling element 499 that is configured to mate or engage with the at least one first coupling element 420 of the retractor component 410 to facilitate the attachment of the attachment component 450 to the retractor component 410.

The attachment component 450 comprises a main body portion 451 comprising a first end 452, a second end 453, and a through-hole 454 that extends from the first end 452 to the second end 453 along a hole axis A-A. In the exemplified embodiment, the at least one second coupling element 499 comprises a first slot 455 formed into an upper portion of the main body portion 451 and a second slot 456 formed into a lower portion of the main body portion 451. The first and second slots 455, 456 may form a pair of slots. The first and second slots 455, 456 may each form an aperture that extends through the thickness of the main body portion 451 from an outer surface thereof to the through-hole 454. The first and second slots 455, 456 are located adjacent to the first end 452 of the main body portion 451 of the attachment component 450.

In the exemplified embodiment, the first and second slots 455, 456 are located on the upper and lower portions of the main body portion 451, respectively. In an alternative embodiment, the first and second slots 455, 456 may be located in first and second side portions of the main body portion 451. In still other embodiments, there may be slots located along each of the upper, lower, first side, and second side portions of the main body portion 451. Each of the slots may be positioned and arranged to correspond to one of the tabs 421, 422 of the retractor component 410.

The retractor component 410 is configured to be detachably coupled to the attachment component 450 via engagement between the at least one first coupling element 420 of the retractor component 410 and the at least one second coupling element 499 of the attachment component 450. Specifically, in the exemplified embodiment the detachable coupling is achieved via engagement between the first tab 421 of the retractor component 410 with the first slot 455 of the attachment component 450 and engagement between the second tab 422 of the retractor component 410 and the second slot 456 of the attachment component 450. In the exemplified embodiment, the first tab 421 nests within the first slot 455 and the second tab 422 nests within he second slot 456 when the retractor component 410 is coupled to the attachment component 450. A user may be able to detach the retractor component 410 from the attachment component 450 by squeezing the retractor component 410, thereby pulling the first and second tabs 421, 422 out of the first and second slots 455, 456, and then pulling the retractor component 410 and the attachment component 450 apart. This attachment/detachment can be achieved regardless of whether the tabs/slots are located on the upper/lower portions of the components or on the side portions of the components, or both. Furthermore, in an alternative embodiment the at least one first coupling element 420 of the retractor component 410 may comprise the slots and the at least one second coupling element 499 of the attachment component 450 may comprise the tabs that nest within the slots when the retractor and attachment components 410, 450 are coupled together.

In the exemplified embodiment, the retractor component 410 and the attachment component 450 are detachably coupled together so that when they are attached, relative movement between them is precluded. Specifically, when the retractor and attachment components 450 are coupled together, movement of either of the retractor component 410 and the attachment component 450 in any direction will result in the other one of the retractor and attachment components 450 also moving in that same direction. In some embodiments, such movement may occur while the retractor component 410 is located in the user’s mouth to retract the user’s cheeks away from the user’s teeth during a treatment session. If the user moves their head during the treatment session, the retractor component 410 and the attachment component 450 will collectively move along with the user’s head. This may ensure that any light treatment being applied by the whitening device 100 will continue to be applied onto the proper parts of the user’s mouth – specifically, the teeth. In other words, this will help to ensure that the light being emitted by the whitening device 100 is not accidentally applied onto the user’s cheeks or other soft tissue surfaces, which could create an undesirable result (such as a burn on the inner surfaces of the user’s cheek if the light from the potentially high power LEDs is applied onto the inner surfaces of the user’s cheek for an extended period of time).

The attachment component 450 further comprises the attachment portion 498, which is configured to facilitate the attachment of the attachment component 450 to the whitening device 100. The attachment portion 498 of the attachment component 450 may comprise a support element 460 that extends from the second end 453 of the main body portion 451. The support element 460 may extend from the second end 453 of the main body portion 451 along a bottom portion of the main body portion 451 that is located below or beneath the through-hole 454. The support element 460 may comprise a top surface 461 and a bottom surface 462 opposite the top surface 461. The top surface 461 may be continuous with a lower surface portion or floor 457 of an inner surface 458 of the main body portion 451.

The attachment portion 498 of the attachment component 450 may further comprise a first engagement tab 463 and a second engagement tab 464. The first and second engagement tabs 463, 464 may be located on opposite sides of the hole axis A-A. More specifically, the first engagement tab 463 may protrude or extend from the support element 460 along or adjacent to a first side edge portion of the support element 460 and the second engagement tab 464 may protrude or extend from the support element 460 along or adjacent to a second side edge portion of the support element 460. In the exemplified embodiment, the first and second engagement tabs 463, 464 may extend from the support element 460 in an upward directly, with the first and second engagement tabs 463, 464 extending from and away from the top surface 461 of the support element 460.

The first engagement tab 463 comprises an inner surface 465 and an outer surface 466. The second engagement tab 464 comprises an inner surface 467 and an outer surface 468. The inner surfaces 465, 467 of the first and second engagement tabs 463, 464 may face one another and the outer surface 466, 468 of the first and second engagement tabs 463, 464 may face outwardly away from one another. In an embodiment, the inner surfaces 465, 467 of the first and second engagement tabs 463, 464 may be concave. In an embodiment, the outer surfaces 466, 468 of the first and second engagement tabs 463, 464 may be convex. The first and second engagement tabs 464, 465 may be spaced apart from the second end 453 of the main body portion 451 of the attachment component 450.

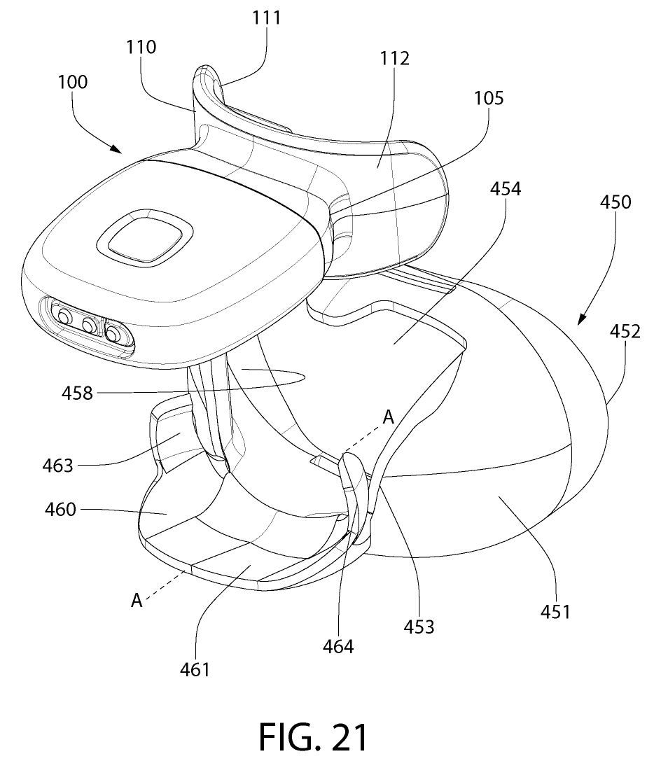

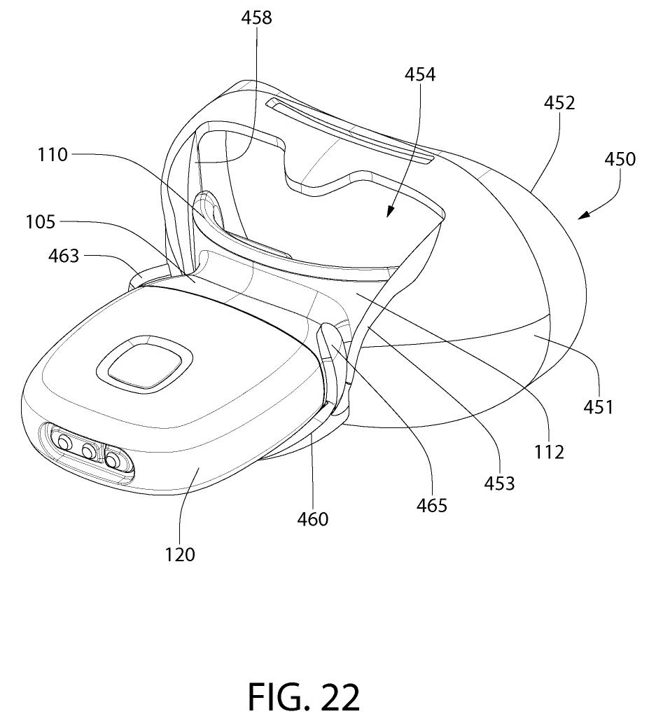

Referring to FIGS. 21 and 22, the attachment of the whitening device 100 to the cheek retractor assembly 400 is illustrated. In FIGS. 21 and 22, the attachment component 450 of the cheek retractor device 400 is illustrated without the retractor component 410 coupled thereto. However, the retractor component 410 and the attachment component 450 may already be coupled together when the whitening device 100 is coupled thereto. In an embodiment, the whitening device 100 may be coupled to the cheek retractor assembly 400 by moving the whitening device 100 downwardly towards the cheek retractor assembly 400 in a direction that is perpendicular to the hole axis A-A. The whitening device 100 is moved downwardly so that the mouthpiece 110 of the whitening device 100 passes through a hole in the top end of the attachment component 450 and into the hollow interior of the attachment component 450 that is defined by the through-hole 454. As the mouthpiece 110 of the whitening device 100 is moved into the hollow interior of the attachment component 450, the rear surface 112 of the mouthpiece 110 comes into abutting contact with the inner surface 458 of the main body portion 451 at a location adjacent to the second end 453 of the main body portion 451.

At the same time, as the whitening device 100 is moved downwardly towards the attachment component 450, the first and second engagement tabs 463, 465 of the attachment portion 498 of the attachment component 410 come into engagement with opposing sides of the transition portion 105 of the whitening device 100. The concave inner surfaces 465, 467 of the first and second engagement tabs 463, 464 engage with the annular concave portion of the outer surface of the whitening device 100 formed by the transition portion 105. The first and second engagement tabs 463, 464 partially wrap around the transition portion 105 from the opposing sides thereof to extend partially over the top thereof. This may prevent the whitening device 100 from being separated from the cheek retractor assembly 400 by moving the whitening device 100 upwardly relative to the cheek retractor assembly 400 unless purposefully done by the user. Furthermore, the whitening device 100 cannot move in either direction parallel to the hole axis A-A. Specifically, the whitening device 100 cannot move in the direction of the second end 453 of the main body portion 451 of the attachment component 450 due to the abutment of the rear surface 112 of the mouthpiece 110 with the inner surface 458 of the attachment component 450. The whitening device 100 cannot move in the direction of the first end 452 of the main body portion 451 due to the engagement between the first and second engagement tabs 463, 464 and the outer surface of the whitening device 100.

When the whitening device 100 is coupled to the cheek retractor assembly 400 (and more specifically to the attachment component 450 thereof), the mouthpiece 110 of the whitening device 100 is located at least partially within the through-hole 454 and the handle 120 of the whitening device 100 protrudes from the second end 453 of the main body portion 451 of the attachment component 450. Furthermore, the handle 120 of the whitening device 100 rests atop of and is supported by the support element 460 of the attachment component 450. This ensures that the whitening device 100 is balanced since a majority of its weight is in the handle 120.

When the whitening device 100 is coupled to the cheek retractor assembly 400 as shown in FIG. 22, relative movement between the whitening device 100 and the cheek retractor assembly 400 is precluded. Thus, if the cheek retractor assembly 400 is at least partially positioned in a user’s mouth and the whitening device 100 is coupled thereto, movement of the user’s head will cause the cheek retractor assembly 400 and the whitening device 100 to move collectively in tandem along with the user’s head. This will ensure that the light emitted from the whitening device 100 is transmitted onto the user’s teeth and not onto the user’s gums, inner cheek surfaces, or the like where the light could cause injury to the user. The cheek retractor assembly 400 will not be capable of moving without the whitening device 100 moving along with it. Even for a slight movement of the check retractor assembly 400, the whitening device 100 will move along with the cheek retractor assembly 400.

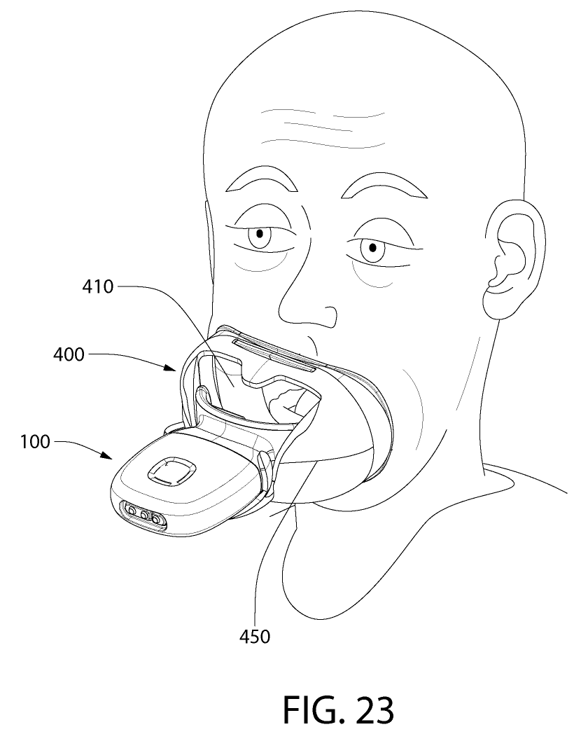

FIG. 23 illustrates the tooth whitening system in use whereby the retractor component 410 of the cheek retractor assembly 400 is positioned within a user’s mouth, the attachment component 450 is detachably coupled to the retractor component 410, and the whitening device 100 is coupled to the attachment component 450. When the whitening device 100 is used in conjunction with the cheek retractor assembly 400, the whitening device 100 is entirely located exterior to the user’s mouth. As such, the bite platform previously described is not actually bitten by the user or located between the user’s teeth, but is instead located wholly exterior to the user’s mouth. However, the whitening device 100 is positioned so that the light emitted therefrom is emitted onto the user’s teeth which are exposed due to the retractor component 410 of the cheek retractor assembly 400 retracting the user’s cheeks away from the user’s teeth. As has been described above, if the user were to move his or her head, the cheek retractor assembly 400 and the whitening device 100 will move along with the user’s head so that the light emitted from the light emitters of the whitening device 100 remains being transmitted only onto the user’s teeth and not onto the user’s inner cheek surfaces where the light could injure the user.

In some embodiments, the whitening device 100 may be powered by the power cord 300 when used with the cheek retractor assembly 400. However, this is not required in all embodiments and the whitening device 100 may be powered by its internal power source 155 without the power cord 300 and still be used with the cheek retractor assembly 400.

In an embodiment, the whitening device 100 may be a small, portable device that can fit easily inside of a user’s single hand and be carrier on the user’s person. The whitening device 100 is capable of being used anywhere: at home, in the user’s office, in a dental office, on the subway, inside, outside, etc. This is because the whitening device 100 is not affixed to any structure. This enables the whitening device 100 to be used in the dentist’s office (with or without the power cord 300) and also to be used at home by the user without dentist supervision. The ability to use such a portable device with the cheek retractor assembly 400 enhances the offerings provided by the dentist. For example, as noted herein when the whitening device 100 is used with the power cord 300, the whitening device 100 may emit light with a greater power output than when used without the power cord 300. Thus, a dentist may help a patient to use the whitening device 100 with the power cord 300 and with the cheek retractor assembly 400 to ensure proper use and positioning of the whitening device 100 when being used with high power due to the power cord 300. Then, when the user takes the whitening device 100 home, the user can use the whitening device 100 at home without the power cord 300 so that the light is emitted with a lower power output with less risk of injury, damage, burns to the patient’s inner cheek and other soft tissue surfaces. The whitening device 100 has a greater flexibility in its use possibilities while providing enhanced whitening benefits to a patient or user.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

While the invention has been described with respect to specific examples including presently preferred modes of carrying out the invention, those skilled in the art will appreciate that there are numerous variations and permutations of the above described systems and techniques. It is to be understood that other embodiments may be utilized and structural and functional modifications may be made without departing from the scope of the present invention. Thus, the spirit and scope of the invention should be construed broadly as set forth in the appended claims.

### EXEMPLARY CLAIMS

The following is a non-limiting listing of exemplary claims:

Exemplary Claim 1. A tooth whitening system comprising: a whitening device comprising: a mouthpiece comprising a front surface; a light emitting assembly comprising one or more light emitters that are configured to generate light which is emitted from the front surface of the mouthpiece; a control circuit comprising a power source that is operably coupled to the light emitting assembly and an actuator; a power cord having a first plug configured to be operably coupled to an external power supply and a second plug configured to be operably coupled to the whitening device; wherein in a first activation state, the actuator is activated without the power cord being coupled to the whitening device and to the external power supply and the light emitting assembly is powered by the power source of the control circuit; and wherein in a second activation state, the actuator is activated with the power cord coupled to the external power supply and to the whitening device and the light emitting assembly is powered by the external power supply.

Exemplary Claim 2. The tooth whitening system according to exemplary claim 1 wherein the power cord does not charge the power source of the control circuit.

Exemplary Claim 3. The tooth whitening system according to exemplary claim 1 or exemplary claim 2 wherein in the first activation state the one or more light emitters are activated at a first current and in the second activation state the one or more light emitters are activated at a second current, the second current being greater than the first current.

Exemplary Claim 4. The tooth whitening system according to any one of exemplary claims 1 to 3 wherein in the second activation state, the power source of the control circuit is bypassed and the light emitting assembly is powered by the external power supply.

Exemplary Claim 5. The tooth whitening system according to any one of exemplary claims 1 to 4 further comprising: the control circuit comprising a first current drive circuit that is operably coupled to the power source to provide a first current to the one or more light emitters in the first activation state; and the control circuit comprising a second current drive circuit that provides a second current to the one or more light emitters in the second activation state, the second current drive circuit being distinct from the first current drive circuit.

Exemplary Claim 6. The tooth whitening system according to exemplary claim 5 wherein the second current is a higher current than the first current.

Exemplary Claim 7. The tooth whitening system according to any one of exemplary claims 1 to 6 further comprising: a charging device configured to be operably coupled to the whitening device to charge the power source of the control circuit.

Exemplary Claim 8. The tooth whitening system according to exemplary claim 7 wherein only one of the charging device and the power cord are able to be coupled to the whitening device at a time.

Exemplary Claim 9. The tooth whitening system according to any one of exemplary claims 7 to 8 further comprising: the whitening device comprising a first electrical contact that is operably coupled to the light emitting assembly, a second electrical contact that is operably coupled to the power source of the control circuit, and a third electrical contact that is operably coupled to ground; the second plug of the power cord comprising a fourth electrical contact that engages the first electrical contact of the whitening device and a fifth electrical contact that engages the third electrical contact of the whitening device when the second plug of the power cord is coupled to the whitening device; and the charging device comprising a sixth electrical contact that engages the second electrical contact of the whitening device and a seventh electrical contact that engages the third electrical contact of the whitening device when the charging device is operably coupled to the whitening device.

Exemplary Claim 10. The tooth whitening system according to any one of exemplary claims 1 to 9 wherein the one or more light emitters comprises a plurality of light emitting diodes, and wherein each of the plurality of light emitting diodes is configured to generate light having a wavelength in a range between 380nm and 440nm.

Exemplary Claim 11. The tooth whitening system according to exemplary claim 10 wherein the wavelength is a range between 405nm and 415nm.

Exemplary Claim 12. The tooth whitening system according to any one of exemplary claims 1 to 11 further comprising: the whitening device comprising: a housing coupled to the mouthpiece and extending from a rear surface of the mouthpiece that is opposite the front surface of the mouthpiece, wherein the housing comprises a cavity and the control circuit is at least partially located within the cavity.

Exemplary Claim 13. The tooth whitening system according to any one of exemplary claims 1 to 12 further comprising: the front surface of the mouthpiece being concave, the mouthpiece further comprising a bite platform protruding from the front surface of the mouthpiece.

Exemplary Claim 14. A tooth whitening system comprising: a whitening device comprising: a mouthpiece comprising a front surface; a light emitting assembly comprising one or more light emitters that are configured to generate light which is emitted from the front surface of the mouthpiece; a control circuit comprising a power source that is operably coupled to the light emitting assembly and an actuator; a power cord having a first plug configured to be operably coupled to an external power supply and a second plug configured to be operably coupled to the whitening device; wherein in a first activation state, the actuator is activated without the power cord being coupled to the whitening device and to the external power supply and the one or more light emitters are configured to generate the light at a first power; wherein in a second activation state, the actuator is activated with the power cord coupled to the external power supply and to the whitening device and the one or more light emitters are configured to generate light at a second power output, the second power output being greater than the first power output.

Exemplary Claim 15. The tooth whitening system according to exemplary claim 14 further comprising: wherein in the first activation state the one or more light emitters are powered solely by the power source of the control circuit; and wherein in the second activation state the one or more light emitters are powered solely by the external power supply, thereby bypassing the power source of the control circuit.

Exemplary Claim 16. The tooth whitening system according to any one of exemplary claims 14 to 15 wherein the power cord does not charge the power source of the control circuit.

Exemplary Claim 17. The tooth whitening system according to any one of exemplary claims 14 to 16 further comprising: the control circuit comprising a first current drive circuit that is operably coupled to the power source to provide a first current to the one or more light emitters in the first activation state; and the control circuit comprising a second current drive circuit that provides a second current to the one or more light emitters in the second activation state, the second current drive circuit being distinct from the first current drive circuit, and the second current being greater than the first current.

Exemplary Claim 18. The tooth whitening system according to any one of exemplary claims 14 to 17 further comprising: a charging device configured to be operably coupled to the whitening device to charge the power source of the control circuit; and wherein only one of the charging unit and the power cord are able to be coupled to the whitening device at a time.

Exemplary Claim 19. The tooth whitening system according to exemplary claim 18 further comprising: the whitening device comprising a first electrical contact that is operably coupled to the light emitting assembly, a second electrical contact that is operably coupled to the power source of the control circuit, and a third electrical contact that is operably coupled to ground; the second plug of the power cord comprising a fourth electrical contact that engages the first electrical contact of the whitening device and a fifth electrical contact that engages the third electrical contact of the whitening device when the second plug of the power cord is coupled to the whitening device; and the charging device comprising a sixth electrical contact that engages the second electrical contact of the whitening device and a seventh electrical contact that engages the third electrical contact of the whitening device when the charging device is operably coupled to the whitening device.

Exemplary Claim 20. A tooth whitening kit comprising: a whitening device comprising: a mouthpiece comprising a front surface; a light emitting assembly comprising one or more light emitters that are configured to generate light which is emitted from the front surface of the mouthpiece; a control circuit comprising: a power source that is operably coupled to the light emitting assembly; an actuator; a first electrical contact that is operably coupled to the light emitting assembly; a second electrical contact that is operably coupled to the power source of the control circuit; and a third electrical contact that is operably coupled to ground; a power cord having a fourth electrical contact and a fifth electrical contact, the fourth and fifth electrical contacts configured to be operably coupled to the first and third electrical contacts of the control circuit, respectively, when the power cord is plugged into the whitening device; and a charging device having a sixth electrical contact and a seventh electrical contact, the sixth and seventh electrical contacts configured to be operably coupled to the second and third electrical contacts of the control circuit, respectively, when the whitening device is being charged by the charging device.

Exemplary Claim 21. A tooth whitening kit comprising: a package containing: a tooth whitening device having a power source and one or more light emitters that are operably coupled to the power source, wherein in a first activation state the one or more light emitters are configured to be powered by the power source so that the one or more light emitters generate light; a charging device configured to be operably coupled to the tooth whitening device to charge the power source of the tooth whitening device; and a power cord configured to be operably coupled to an external power supply and to the tooth whitening device so that in a second activation state the one or more light emitters are configured to be powered by the external power supply via the power cord to generate the light while bypassing the power source.

Exemplary Claim 22. The tooth whitening kit according to exemplary claim 21 further comprising: the package further containing: a cheek retractor assembly that is configured to be coupled to the tooth whitening device; at least one first dispensing unit configured to dispense a gingival barrier composition; and at least one second dispensing unit configured to dispense a whitening composition.

Exemplary Claim 23. The tooth whitening kit according to exemplary claim 22 wherein the at least one second dispensing unit is a dual barrel syringe and wherein the whitening composition comprises a peroxide component in a first chamber of the dual barrel syringe and a pH component in a second chamber of the dual barrel syringe.

Exemplary Claim 24. The tooth whitening kit according to exemplary claim 23 further comprising: the package further containing: at least one container receiving the whitening composition; and at least one applicator for applying the whitening component onto a user’s teeth.

Exemplary Claim 25. The whitening kit according to any one of exemplary claims 22 to 24 wherein the cheek retractor assembly comprises a retractor component that is configured to be placed inside of a user’s mouth to retract the user’s cheeks and an attachment component that is configured to facilitate an attachment of the tooth whitening device to the retractor component.

Exemplary Claim 26. A tooth whitening method comprising: performing a first tooth whitening session under dentist supervision, the first tooth whitening session comprising: positioning a mouthpiece of a whitening device into or adjacent to a user’s mouth; operably coupling the whitening device to an external power supply via a power cord; activating the whitening device so that light emitters of the whitening device generate light that is emitted from the mouthpiece and onto the user’s teeth, the light emitters being powered by the external power supply; and deactivating the whitening device after elapse of a first period of time; performing a second tooth whitening session without dentist supervision, the second tooth whitening session comprising: positioning the mouthpiece of the whitening device into or adjacent to the user’s mouth; activating the whitening device so that the light emitters of the whitening device generate light that is emitted from the mouthpiece and onto the user’s teeth, the light emitters being powered by a power source of the whitening device; and deactivating the whitening device after elapse of a second period of time.

Exemplary Claim 27. The tooth whitening method according to exemplary claim 26 wherein during the first tooth whitening session the light emitters generate the light at a first power output, and wherein during the second tooth whitening session the light emitters generate the light at a second power output that is less than the first power output.

Exemplary Claim 28. The tooth whitening method according to exemplary claim 26 or exemplary claim 27 wherein during the first tooth whitening session the light emitters are driven by a first current and wherein during the second tooth whitening session the light emitters are driven by a second current that is less than the first current.

Exemplary Claim 29. The tooth whitening method according to any one of exemplary claims 26 to 28 further comprising: in both of the first and second tooth whitening sessions, either prior to or simultaneous with the positioning of the mouthpiece of the whitening device into or adjacent to the user’s mouth, applying a tooth whitening composition onto the user’s teeth.

Exemplary Claim 30. The tooth whitening method according to any one of exemplary claims 26 to 29 wherein during the first tooth whitening session the power source of the whitening device is not charged by the power cord.

Exemplary Claim 31. The tooth whitening method according to exemplary claim 30 wherein the power source of the whitening device comprises batteries that are housed within a housing of the whitening device, and wherein the external power supply is selected from the group consisting of a mains power supply and an external electronic device.

Exemplary Claim 32. The tooth whitening method according to any one of exemplary claims 26 to 31 further comprising: in the first tooth whitening session, prior to positioning the mouthpiece of the whitening device into or adjacent to the user’s mouth: positioning a cheek retractor into the user’s mouth to retract the user’s cheeks away from the user’s teeth; and coupling the tooth whitening device to the cheek retractor so that relative movement between the cheek retractor and the tooth whitening device is precluded.

Exemplary Claim 33. The tooth whitening method according to exemplary claim 32 further comprising the mouthpiece being located external to the user’s mouth during the first tooth whitening session.

Exemplary Claim 34. The tooth whitening method according to exemplary claim 32 or exemplary claim 33 further comprising: in the second tooth whitening session, positioning the mouthpiece of the whitening device into or adjacent to the user’s mouth comprises placing the mouthpiece at least partially inside of the user’s mouth.

Exemplary Claim 35. A tooth whitening system comprising: a whitening device comprising: a mouthpiece comprising a front surface and a rear surface opposite the front surface; and a handle protruding from the rear surface; and a first attachment portion; a cheek retractor assembly comprising: a cheek retractor portion that is configured to be positioned within a user’s mouth to retract the user’s cheeks; and a second attachment portion; wherein the whitening device is configured to be detachably coupled to the cheek retractor assembly via engagement between the second attachment portion of the cheek retractor assembly and the first attachment portion of the whitening device; and wherein when the whitening device is coupled to the cheek retractor assembly, relative movement between the whitening device and the cheek retractor assembly is precluded.

Exemplary Claim 36. The tooth whitening system according to exemplary claim 35 further comprising: the cheek retractor assembly comprising: a retractor component that comprises the cheek retractor portion and at least one first coupling element; and an attachment component that comprises the second attachment portion and at least one second coupling element; and wherein the retractor component is detachably coupled to the attachment component via engagement between the at least one first coupling element of the retractor component and the at least one second coupling element of the attachment component such that relative movement between the retractor and attachment components is precluded.

Exemplary Claim 37. The tooth whitening system according to exemplary claim 36 further comprising: the at least one first coupling element comprising a first one of a pair of slots and a pair of tabs; the at least one second coupling element comprising a second one of the pair of slots and the pair of tabs, one of the first and second ones being the pair of slots and the other one of the first and second ones being the pair of tabs; and wherein when the retractor component is coupled to the attachment component, the pair of tabs extend into the pair of slots to couple the attachment component to the retractor component.

Exemplary Claim 38. The tooth whitening system according to exemplary claim 36 or exemplary claim 37 further comprising: the attachment component comprising: a main body portion comprising a first end, a second end, and a through-hole that extends from the first end to the second end, the at least one second coupling element being located adjacent to the first end; and a support element extending from the second end of the main body portion; and wherein when the whitening device is coupled to the cheek retractor assembly, at least a portion of the mouthpiece of the whitening device is located within the through-hole of the main body portion of the attachment component and at least a portion of the housing of the whitening device rests atop of the support element of the attachment component.

Exemplary Claim 39. The tooth whitening system according to exemplary claim 38 further comprising: the through-hole extending along a hole axis from a first opening in the first end of the main body portion to a second opening in the second end of the main body portion; the second attachment portion comprising a first attachment tab protruding upwardly from the support element on a first side of the hole axis and a second attachment tab protruding upwardly from the support element on a second side of the hole axis; and wherein the first and second engagement tabs of the second attachment portion engage a portion of the outer surface of the whitening device to facilitate an attachment of the whitening device to the cheek retractor assembly.

Exemplary Claim 40. The tooth whitening system according to exemplary claim 39 further comprising: the outer surface of the whitening device comprising a concave portion that forms the first attachment portion of the whitening device; and wherein the first and second engagement tabs comprise concave interior surfaces that directly contact the concave portion of the outer surface of the whitening device to facilitate the coupling of the whitening device to the cheek retractor assembly.

Exemplary Claim 41. The tooth whitening system according to any one of exemplary claims 35 to 40 further comprising: the cheek retractor assembly comprising a hollow interior; and wherein when the whitening device is coupled to the cheek retractor assembly, the mouthpiece of the whitening device is located within the hollow interior of the cheek retractor assembly and the handle of the whitening device protrudes from an end of a main body portion of the cheek retractor assembly.

Exemplary Claim 42. The tooth whitening system according to exemplary claim 41 further comprising: the cheek retractor assembly comprising a support element protruding from the end of the main body portion of the cheek retractor assembly, and wherein at least a portion of the handle of the whitening device rests atop of the support element when the whitening device is coupled to the cheek retractor assembly.

Exemplary Claim 43. The tooth whitening system according to exemplary claim 41 or exemplary claim 42 wherein when the whitening device is coupled to the cheek retractor assembly, a portion of the rear surface of the mouthpiece abuts against a portion of an inner surface of the cheek retractor assembly.

Exemplary Claim 44. The tooth whitening system according to any one of exemplary claims 35 to 43 further comprising: the whitening device being configured to be used in a first type of tooth whitening session wherein the whitening device is physically coupled to the cheek retractor assembly and the cheek retractor assembly is at least partially positioned in the user’s mouth to retract the user’s teeth, the whitening device being located external to the user’s mouth in the first type of tooth whitening session; and the whitening device being configured to be used in a second type of tooth whitening session wherein the mouthpiece of the whitening device is located inside the user’s mouth, the cheek retractor not being used to retract the user’s teeth in the second type of tooth whitening session.

Exemplary Claim 45. The tooth whitening system according to any one of exemplary claims 35 to 44 wherein the front surface of the mouthpiece is concave and the rear surface of the mouthpiece is convex.

Exemplary Claim 46. The tooth whitening system according to any one of exemplary claims 35 to 45 further comprising: the whitening device comprising a light emitting assembly comprising one or more light emitters that are configured to generate light which is emitted from the front surface of the mouthpiece; and the handle comprising a cavity that houses a power source that is operably coupled to the light emitting assembly to power the light emitters when activated in a first activation state.

Exemplary Claim 47. The tooth whitening system according to exemplary claim 46 further comprising: a power cord having a first plug configured to be operably coupled to an external power supply and a second plug configured to be operably coupled to the whitening device; wherein in a first activation state, the actuator is activated without the power cord being coupled to the whitening device and to the external power supply and the one or more light emitters are powered by the power source of the whitening device; and wherein in a second activation state, the actuator is activated with the power cord coupled to the external power supply and to the whitening device and the light emitting assembly is powered by the external power supply.

Exemplary Claim 48. The tooth whitening system according to exemplary claim 47 further comprising: wherein in the first activation state the one or more light emitters are configured to generate the light at a first power output; and wherein in the second activation state the one or more light emitters are configured to generate light at a second power output, the second power output being greater than the first power output.

Exemplary Claim 49. The tooth whitening system according to any one of exemplary claims 35 to 48 wherein the cheek retractor assembly comprises a through-hole that extends along longitudinal axis from a first end of the cheek retractor assembly to a second end of the cheek retractor assembly, and wherein the whitening device is configured to be coupled to the cheek retractor assembly by moving the whitening device relative to the cheek retractor assembly in a first direction perpendicular to the longitudinal axis.

Exemplary Claim 50. The tooth whitening system according to exemplary claim 49 wherein when the whitening device is coupled to the cheek retractor assembly, the whitening device is precluded from being detached from the cheek retractor assembly without moving the whitening device relative to the cheek retractor assembly in a second direction perpendicular to the longitudinal axis, the second direction being opposite the first direction.

Exemplary Claim 51. The tooth whitening system according to any one of exemplary claims 35 to 50 further comprising: the first attachment portion being a concave portion of an outer surface of the whitening device formed at a transition between the rear surface of the mouthpiece and the handle; and the second attachment portion comprising a pair of engagement tabs having concave inner surfaces that engage the concave portion of the outer surface of the whitening device.

Exemplary Claim 52. The tooth whitening system according to exemplary claim 51 wherein the concave portion of the outer surface of the whitening device is formed partially by the rear surface of the mouthpiece and partially by a side surface of the handle.

Exemplary Claim 53. A tooth whitening system comprising: a whitening device comprising: a mouthpiece comprising a front surface and a rear surface; a light emitting assembly comprising one or more light emitters that are configured to generate light which is emitted from the front surface of the mouthpiece; and a handle extending from the rear surface; a cheek retractor assembly; wherein the whitening device is configured to be detachably coupled to the cheek retractor assembly such that relative movement between the whitening device and the cheek retractor assembly is precluded.

Exemplary Claim 54. The tooth whitening system according to exemplary claim 53 further comprising: the cheek retractor assembly comprising a main body portion having a first end, a second end, and a through-hole extending from the first end to the second end; and wherein when the whitening device is coupled to the cheek retractor assembly, the mouthpiece is at least partially located within the through-hole of the main body portion of the cheek retractor assembly and the housing of the whitening device protrudes from the second end of the main body portion of the cheek retractor assembly.

Exemplary Claim 55. The tooth whitening system according to exemplary claim 54 further comprising: the cheek retractor assembly comprising a support element extending from the second end of the cheek retractor; and wherein when the whitening device is coupled to the cheek retractor assembly, the handle of the whitening device rests atop of the support element.

Exemplary Claim 56. The tooth whitening system according to exemplary claim 55 further comprising: the cheek retractor assembly comprising a pair of engagement tabs protruding from opposing edges of the support element; and wherein the pair of engagement tabs engage opposing sides of an outer surface of the whitening device to secure the whitening device to the cheek retractor assembly.

Exemplary Claim 57. The tooth whitening system according to any one of exemplary claims 53 to 56 further comprising: the cheek retractor assembly comprising: a retractor component comprising one or more first attachment elements, the retractor component configured to be inserted into a user’s mouth to retract the user’s inner cheek surfaces away from the user’s teeth; and an attachment component comprising one or more second attachment elements; wherein the one or more first attachment elements are configured to mate with the one or more second attachment elements to couple the retractor component to the attachment component, and wherein the whitening device is configured to be detachably coupled to the attachment component of the cheek retractor assembly.

Exemplary Claim 58. The tooth whitening system according to exemplary claim 57 wherein when the retractor component and the attachment component are coupled together, relative movement therebetween is precluded.

What is claimed is:

1. A tooth whitening system comprising:
a whitening device comprising:
a mouthpiece comprising a front surface;
a light emitting assembly comprising one or more light emitters that are configured to generate light which is emitted from the front surface of the mouthpiece;
a control circuit comprising a power source that is operably coupled to the light emitting assembly and an actuator;
a power cord having a first plug configured to be operably coupled to an external power supply and a second plug configured to be operably coupled to the whitening device;
wherein in a first activation state, the actuator is activated without the power cord being coupled to the whitening device and to the external power supply and the light emitting assembly is powered by the power source of the control circuit; and
wherein in a second activation state, the actuator is activated with the power cord coupled to the external power supply and to the whitening device and the light emitting assembly is powered by the external power supply.

2. The tooth whitening system according to claim 1 wherein the power cord does not charge the power source of the control circuit.

3. The tooth whitening system according to claim 1 wherein in the first activation state the one or more light emitters are activated at a first current and in the second activation state the one or more light emitters are activated at a second current, the second current being greater than the first current.

4. The tooth whitening system according to claim 1 wherein in the second activation state, the power source of the control circuit is bypassed and the light emitting assembly is powered by the external power supply.

5. The tooth whitening system according to claim 1 further comprising:
the control circuit comprising a first current drive circuit that is operably coupled to the power source to provide a first current to the one or more light emitters in the first activation state; and
the control circuit comprising a second current drive circuit that provides a second current to the one or more light emitters in the second activation state, the second current drive circuit being distinct from the first current drive circuit.

6. The tooth whitening system according to claim 1 further comprising:
a charging device configured to be operably coupled to the whitening device to charge the power source of the control circuit;
wherein only one of the charging device and the power cord are able to be coupled to the whitening device at a time.

7. The tooth whitening system according to claim 6 further comprising:
the whitening device comprising a first electrical contact that is operably coupled to the light emitting assembly, a second electrical contact that is operably coupled to the power source of the control circuit, and a third electrical contact that is operably coupled to ground;
the second plug of the power cord comprising a fourth electrical contact that engages the first electrical contact of the whitening device and a fifth electrical contact that engages the third electrical contact of the whitening device when the second plug of the power cord is coupled to the whitening device; and
the charging device comprising a sixth electrical contact that engages the second electrical contact of the whitening device and a seventh electrical contact that engages the third electrical contact of the whitening device when the charging device is operably coupled to the whitening device.

8. The tooth whitening system according to claim 1 wherein the one or more light emitters comprises a plurality of light emitting diodes, and wherein each of the plurality of light emitting diodes is configured to generate light having a wavelength in a range between 380nm and 440nm.

9. The tooth whitening system according to claim 1 further comprising:
the whitening device comprising:
a housing coupled to the mouthpiece and extending from a rear surface of the mouthpiece that is opposite the front surface of the mouthpiece, wherein the housing comprises a cavity and the control circuit is at least partially located within the cavity.

10. A tooth whitening system comprising:
a whitening device comprising:
a mouthpiece comprising a front surface;
a light emitting assembly comprising one or more light emitters that are configured to generate light which is emitted from the front surface of the mouthpiece;
a control circuit comprising a power source that is operably coupled to the light emitting assembly and an actuator;
a power cord having a first plug configured to be operably coupled to an external power supply and a second plug configured to be operably coupled to the whitening device;
wherein in a first activation state, the actuator is activated without the power cord being coupled to the whitening device and to the external power supply and the one or more light emitters are configured to generate the light at a first power output;
wherein in a second activation state, the actuator is activated with the power cord coupled to the external power supply and to the whitening device and the one or more light emitters are configured to generate light at a second power output, the second power output being greater than the first power output.

11. The tooth whitening system according to claim 10 further comprising:
wherein in the first activation state the one or more light emitters are powered solely by the power source of the control circuit; and
wherein in the second activation state the one or more light emitters are powered solely by the external power supply, thereby bypassing the power source of the control circuit.

12. The tooth whitening system according to claim 10 wherein the power cord does not charge the power source of the control circuit.

13. The tooth whitening system according to claim 10 further comprising:
the control circuit comprising a first current drive circuit that is operably coupled to the power source to provide a first current to the one or more light emitters in the first activation state; and
the control circuit comprising a second current drive circuit that provides a second current to the one or more light emitters in the second activation state, the second current drive circuit being distinct from the first current drive circuit, and the second current being greater than the first current.

14. The tooth whitening system according to claim 10 further comprising:
a charging device configured to be operably coupled to the whitening device to charge the power source of the control circuit; and
wherein only one of the charging unit and the power cord are able to be coupled to the whitening device at a time.

15. The tooth whitening system according to claim 14 further comprising:
the whitening device comprising a first electrical contact that is operably coupled to the light emitting assembly, a second electrical contact that is operably coupled to the power source of the control circuit, and a third electrical contact that is operably coupled to ground;
the second plug of the power cord comprising a fourth electrical contact that engages the first electrical contact of the whitening device and a fifth electrical contact that engages the third electrical contact of the whitening device when the second plug of the power cord is coupled to the whitening device; and
the charging device comprising a sixth electrical contact that engages the second electrical contact of the whitening device and a seventh electrical contact that engages the third electrical contact of the whitening device when the charging device is operably coupled to the whitening device.

16. A tooth whitening kit comprising:
a package containing:
a tooth whitening device having a power source and one or more light emitters that are operably coupled to the power source, wherein in a first activation state the one or more light emitters are configured to be powered by the power source so that the one or more light emitters generate light;
a charging device configured to be operably coupled to the tooth whitening device to charge the power source of the tooth whitening device; and
a power cord configured to be operably coupled to an external power supply and to the tooth whitening device so that in a second activation state the one or more light emitters are configured to be powered by the external power supply via the power cord to generate the light while bypassing the power source.

17. The tooth whitening kit according to claim 16 further comprising:
the package further containing:
a cheek retractor assembly that is configured to be coupled to the tooth whitening device;
at least one first dispensing unit configured to dispense a gingival barrier composition; and
at least one second dispensing unit configured to dispense a whitening composition.

18. The tooth whitening kit according to claim 17 wherein the at least one second dispensing unit is a dual barrel syringe and wherein the whitening composition comprises a peroxide component in a first chamber of the dual barrel syringe and a pH component in a second chamber of the dual barrel syringe.

19. The tooth whitening kit according to claim 18 further comprising:
the package further containing:
at least one container receiving the whitening composition; and
at least one applicator for applying the whitening component onto a user’s teeth.

20. The tooth whitening kit according to claim 17 wherein the cheek retractor assembly comprises a retractor component that is configured to be placed inside of a user’s mouth to retract the user’s cheeks and an attachment component that is configured to facilitate an attachment of the tooth whitening device to the retractor component.
